(19) United States
(12) Patent Application Publication
(10) Pub. No.: US 2019/0144503 A1
(43) Pub. Date: May 16, 2019

(54) METHODS FOR THE IDENTIFICATION OF BIFUNCTIONAL COMPOUNDS

(71) Applicant: Regents of the University of Minnesota, Minneapolis, MN (US)

(72) Inventor: Roy R. Lobb, Wellesley, MA (US)

(73) Assignee: Regents of the University of Minnesota, Minneapolis, MN (US)

(21) Appl. No.: 16/096,259

(22) PCT Filed: Apr. 27, 2017
(86) PCT No.: PCT/GB2017/051188

§ 371 Date: Oct. 24, 2018

Related U.S. Application Data
(60) Provisional application No. 62/328,355, filed on Apr. 27, 2016.

(51) Int. Cl. C07K 7/54; G01N 33/74; C12Q 1/34; C12Q 1/25; G01N 33/53

(52) U.S. Cl. CPC C07K 7/54; G01N 33/74; C12Y 402/01001; C12Q 1/25; G01N 33/53; C12Q 1/34

(57) ABSTRACT

The present disclosure provides bifunctional compounds including a polypeptide targeting moiety conjugated to a small molecule in a site-specific manner both of which bind to the same target protein resulting in potent and specific binding characteristics and methods of identifying such compounds.

### BACKGROUND

Small molecules can provide unique activity beyond natural amino acids and proteins, for example by binding to active sites (e.g., bioactive pockets) in ion channels, GPCRs, and enzymes. Yet small molecules alone often suffer from a lack of potency, specificity, sub-optimal pharmacokinetic profiles, or other properties. Proteins have been engineered to excel at these challenges yet often lack the ability to potently impact select epitopes. The present disclosure provides compounds and methods which merge the beneficial aspects of small molecules and proteins to develop bifunctional compounds including protein and small molecule binding interfaces as potent, specific agents of extracellular targets.

### SUMMARY

The present disclosure provides bifunctional compounds including a polypeptide targeting moiety conjugated to a small molecule in a site-specific manner, both of which bind to the same target protein resulting in potent and specific binding characteristics, along with methods of identifying such compounds.

Accordingly, in a first aspect, the present disclosure provides a synthetic bifunctional compound, or a pharmaceutically acceptable salt thereof, that modulates the activity of an extracellular target protein (e.g., a soluble protein, a membrane-bound protein, or a transmembrane protein). The bifunctional compound includes a polypeptide targeting moiety (e.g., an antibody or an antigen binding fragment thereof) that binds to the extracellular target protein covalently conjugated to a small molecule moiety (e.g., a sulfonamide-containing moiety, a hydroxamic acid-containing moiety, a thiadiazole sulfonamide-containing moiety, a glutamate-urea-lysine-containing moiety, or a cyclic peptide-containing moiety) that binds to the extracellular target protein, wherein the bifunctional compound binds to the target protein with at least 5-fold greater affinity and/or 5-fold greater selectivity than the affinity of each of the polypeptide targeting moiety and the small molecule moiety alone.

In some embodiments, the polypeptide targeting moiety is an antibody or an antigen binding fragment thereof. In some embodiments, the polypeptide targeting moiety is an antibody. For example, an antibody that binds to a soluble protein, a membrane-bound protein, or a transmembrane protein. In some embodiments, the extracellular target protein is a carbonic anhydrase (e.g., carbonic anhydrase 9 or carbonic anhydrase 2), CXCR4, PSMA, or a metalloprotease. In some embodiments, the antibody is a mAB 38C2 antibody.

In some embodiments, the polypeptide targeting moiety is a fibronectin type III domain, a variable heavy chain (VH) Ab fragment, a single-chain variable fragment, a centyrin, or a DARPin. In some embodiments, the polypeptide targeting moiety is an antigen binding fragment of an antibody conjugated to a second polypeptide (e.g., human aminoguanyltransferase or a distinct variable heavy chain Ab fragment or fibronectin type III domain).

In some embodiments, the polypeptide targeting moiety does not bind at a small molecule binding site (e.g., the active site) of the extracellular target protein. In some embodiments, the polypeptide targeting moiety binds at a small molecule binding site (e.g., the active site) of the extracellular target protein. In some embodiments, the small molecule moiety binds at the active site of the extracellular target protein. In some embodiments, the small molecule moiety does not bind at the active site of the extracellular target protein. In some embodiments, the small molecule moiety and the polypeptide targeting moiety bind to different sites on the extracellular target protein (e.g., the polypeptide targeting moiety does not bind at the active site of the extracellular target protein and the small molecule does bind at the active site of the extracellular target protein).

In some embodiments, the small molecule moiety binds the extracellular target protein with low affinity (e.g., the small molecule binds with a K_{D} greater than 200 nM) when not covalently conjugated to the polypeptide targeting moiety.

In some embodiments, the extracellular target protein belongs to a family of extracellular proteins (e.g., a family of soluble, membrane-bound proteins, or transmembrane protein) and the small molecule moiety binds to at least two members of the family of extracellular proteins. In some embodiments, the small molecule moiety binds to all members of the family of extracellular proteins. In some embodiments, the small molecule moiety binds to at least two members of the family of extracellular proteins with similar affinity (e.g., with less than 5-fold difference in affinity between the at least two members).

In some embodiments, the small molecule moiety is covalently conjugated to the polypeptide targeting moiety via a linker (e.g., via a cysteine, a lysine, or a non-natural amino acid in the polypeptide targeting moiety such as a cysteine, lysine, or non-natural amino acid in CDR1, CDR2, or CDR3 of a variable heavy chain, a framework residue within the binding interface, or a framework residue not within the binding interface). In some embodiments, the small molecule moiety is covalently conjugated to the polypeptide targeting moiety via a free cysteine in the polypeptide targeting moiety (e.g., CDR1, CDR2, or CDR3 of a variable heavy chain) which is formed by reduction of a disulfide bond. In some embodiments, the polypeptide targeting moiety is modified to include a cysteine, lysine, or non-natural amino acid for conjugation to the small molecule moiety. In some embodiments, the small molecule moiety is covalently conjugated to the polypeptide targeting moiety in a site-specific manner. In some embodiments, the site of conjugation is a solvent exposed amino acid of the polypeptide targeting moiety. In some embodiments, the linker includes a protein that does not bind to the extracellular target protein (e.g., human aminoguanyltransferase).

In some embodiments, the linker has the structure of Formula I:

A¹-(B¹)_{f}—(C¹)_{g}—(B²)ₕ-(D)-(B³)ᵢ—(C²)ⱼ—(B⁴)ₖ-A² Formula I

wherein A¹ is a bond between the linker and polypeptide targeting moiety; A² is a bond between the small molecule moiety and the linker; B¹, B², B³, and B⁴ each, independently, is selected from optionally substituted C₁-C₂ alkyl, optionally substituted C₁-C₃ heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, I, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, the bifunctional compound binds to the extracellular target protein pseudo-irreversibly (e.g., the off-rate of the binding of the synthetic bifunctional compound to the extracellular protein is slower than the turnover of the extracellular target protein).

In some embodiments, the synthetic bifunctional compound inhibits the activity of the extracellular target protein (e.g., the compound is an antagonist). In some embodiments, the synthetic bifunctional compound activates the activity of the extracellular target protein (e.g., the compound is an agonist or a partial agonist).

In some embodiments, the extracellular target protein is a druggable target protein. In some embodiments, the extracellular target protein has a small molecule binding site. In some embodiments, the extracellular target protein is a soluble protein. In some embodiments, the extracellular target protein is a membrane bound protein. In some embodiments, the extracellular target protein is a transmembrane protein (e.g., the extracellular domain of a transmembrane protein). In some embodiments, the extracellular target protein is a carbonic anhydrase (e.g., carbonic anhydrase 9 or carbonic anhydrase 2). In some embodiments, the small molecule moiety is a sulfonamide. For example, the small molecule moiety includes the structure of Formula II:

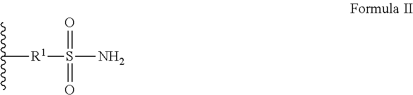

wherein R¹ is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the extracellular target protein is a metalloprotease. In some embodiments, the small molecule moiety is a hydroxamic acid. For example, the small molecule moiety includes the structure of Formula III:

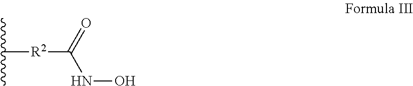

wherein R² is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the extracellular target protein is PSMA. In some embodiments, the small molecule moiety is a thiadiazole sulfonamide or includes a glutamate-urea-lysine moiety. For example, the small molecule moiety includes the structure of Formula IV or Formula V:

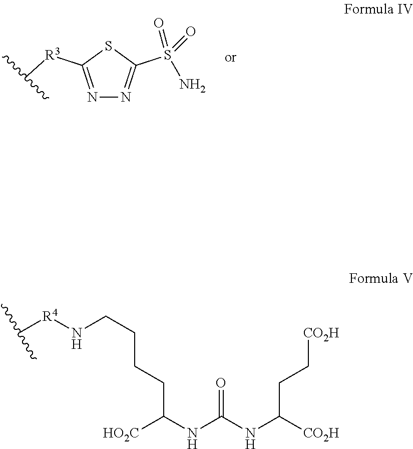

wherein R³ is optionally substituted C₁-C₆ alkyl or optionally substituted C₁-C₆ heteroalkyl (e.g., —NHC(O)—); and

R⁴ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

In some embodiments, the small molecule moiety includes the structure:

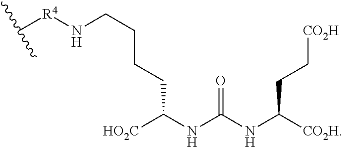

In some embodiments, the extracellular target protein is CXCR4. In some embodiments, the small molecule moiety is a cyclic peptide. For example, the small molecule moiety includes the structure of Formula VI:

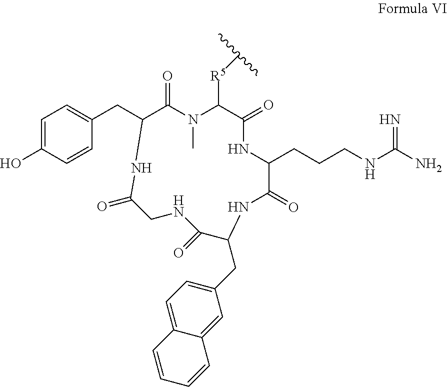

wherein R⁵ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

In some embodiments, the small molecule moiety includes the structure:

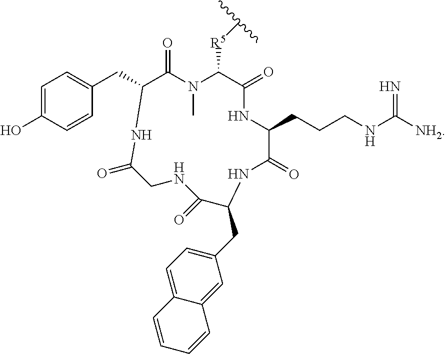

In another aspect, the disclosure provides a method of identifying a compound that modulates the activity of a target protein (e.g., an extracellular protein such as a soluble protein, a membrane-bound protein, or a transmembrane protein). This method includes: (a) providing two or more synthetic bifunctional compounds including a polypeptide targeting moiety (e.g., an antibody or antigen binding fragment thereof) covalently conjugated to a small molecule moiety (e.g., a sulfonamide-containing moiety or a hydroxamic acid-containing moiety); (b) contacting a target protein with the two or more synthetic bifunctional compounds; and (c) determining the binding of the two or more synthetic bifunctional compounds to the target protein, wherein a compound is identified as modulating the activity of the target protein if the synthetic bifunctional compound binds to the target protein with at least 5-fold greater affinity and/or 5-fold greater selectivity than the affinity of each of the polypeptide targeting moiety and the small molecule alone.

In some embodiments, the small molecule moiety of each of the two or more synthetic bifunctional compounds is the same. In some embodiments, the polypeptide targeting moiety of each of the two or more synthetic bifunctional compounds is the same.

In some embodiments, the polypeptide targeting moiety is a fibronectin type III domain, a variable heavy chain (VH) Ab fragment, a single-chain variable fragment, a centyrin, or a DARPin. In some embodiments, the polypeptide targeting moiety is an antigen binding fragment of an antibody conjugated to a second polypeptide (e.g., human aminoguanyltransferase or a distinct variable heavy chain Ab fragment or fibronectin type III domain).

In some embodiments, the polypeptide targeting moiety does not bind at a small molecule binding site (e.g., the active site) of the extracellular target protein. In some embodiments, the polypeptide targeting moiety binds at a small molecule binding site (e.g., the active site) of the extracellular target protein. In some embodiments, the small molecule moiety binds at the active site of the extracellular target protein. In some embodiments, the small molecule moiety does not bind at the active site of the extracellular target protein. In some embodiments, the small molecule moiety and the polypeptide targeting moiety bind to different sites on the extracellular target protein (e.g., the polypeptide targeting moiety does not bind at the active site of the extracellular target protein and the small molecule does bind at the active site of the extracellular target protein).

In some embodiments, the small molecule moiety binds the target protein with low affinity (e.g., the small molecule moiety binds with a K_{D} greater than 200 nM) when not covalently conjugated to the polypeptide targeting moiety.

In some embodiments, the extracellular target protein belongs to a family of extracellular proteins (e.g., a family of soluble, membrane-bound proteins, or transmembrane proteins) and the small molecule moiety binds to at least two members of the family of extracellular proteins. In some embodiments, the small molecule moiety binds to all members of the family of extracellular proteins. In some embodiments, the small molecule moiety binds to at least two members of the family of extracellular proteins with similar affinity (e.g., with less than 5-fold difference in affinity between the at least two members).

In some embodiments, the small molecule moiety is covalently conjugated to the polypeptide targeting moiety via a linker (e.g., via a cysteine, a lysine, or a non-natural amino acid in the polypeptide targeting moiety such as a cysteine, lysine, or non-natural amino acid in CDR1, CDR2, or CDR3 of a variable heavy chain, a framework residue within the binding interface, ora framework residue not within the binding interface). In some embodiments, the polypeptide targeting moiety is modified to include a cysteine, lysine, or non-natural amino acid for conjugation to the small molecule moiety. In some embodiments, the small molecule moiety is covalently conjugated to the polypeptide targeting moiety in a site-specific manner. In some embodiments, the linker includes a protein that does not bind to the extracellular target protein (e.g., human aminoguanyltransferase).

In some embodiments, the linker has the structure of Formula I:

A¹-(B¹)_{f}—(C¹)_{g}—(B²)ₕ-(D)-(B³)ᵢ—(C²)ⱼ—(B⁴)ₖ-A² Formula I

wherein A¹ is a bond between the linker and polypeptide targeting moiety; A² is a bond between the small molecule moiety and the linker; B¹, B², B³, and B⁴ each, independently, is selected from optionally substituted C₁-C₂ alkyl, optionally substituted C₁-C₃ heteroalkyl, O, S, and NR^{N}; R^{N} is hydrogen, optionally substituted C₁₋₄alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, I, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, the bifunctional compound binds to the extracellular target protein pseudo-irreversibly (e.g., the off-rate of the binding of the synthetic bifunctional compound to the extracellular protein is slower than the turnover of the extracellular target protein).

In some embodiments, the synthetic bifunctional compound inhibits the activity of the extracellular target protein (e.g., the compound is an antagonist). In some embodiments, the synthetic bifunctional compound activates the activity of the extracellular target protein (e.g., the compound is an agonist or a partial agonist).

In some embodiments, the extracellular target protein is a druggable target protein. In some embodiments, the extracellular target protein has a small molecule binding site. In some embodiments, the extracellular target protein is a soluble protein. In some embodiments, the extracellular target protein is a membrane bound protein. In some embodiments, the extracellular target protein is a transmembrane protein (e.g., the extracellular domain of a transmembrane protein). In some embodiments, the extracellular target protein is a carbonic anhydrase (e.g., carbonic anhydrase 9 or carbonic anhydrase 2).

In some embodiments, the small molecule moiety is a sulfonamide. For example, the small molecule moiety includes the structure of Formula II:

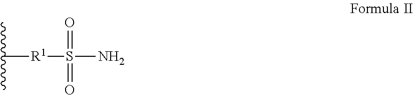

wherein R¹ is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the extracellular target protein is a metalloprotease. In some embodiments, the small molecule moiety is a hydroxamic acid. For example, the small molecule moiety includes the structure of Formula III:

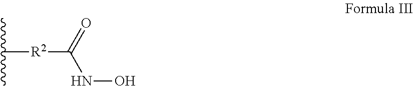

wherein R² is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the extracellular target protein is PSMA. In some embodiments, the small molecule moiety is a thiadiazole sulfonamide or includes a glutamate-urea-lysine moiety. For example, the small molecule moiety includes the structure of Formula IV or Formula V:

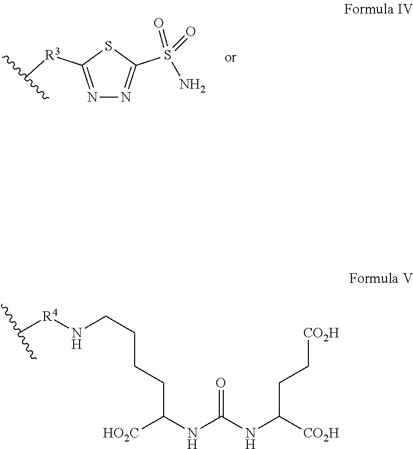

wherein R³ is optionally substituted C₁-C₆ alkyl or optionally substituted C₁-C₆ heteroalkyl (e.g., —NHC(O)—); and

R⁴ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

In some embodiments, the small molecule moiety includes the structure:

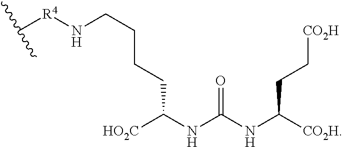

In some embodiments, the extracellular target protein is CXCR4. In some embodiments, the small molecule moiety is a cyclic peptide. For example, the small molecule moiety includes the structure of Formula VI:

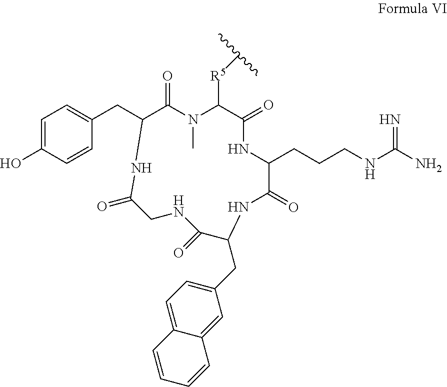

wherein R⁵ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

In some embodiments, the small molecule moiety includes the structure:

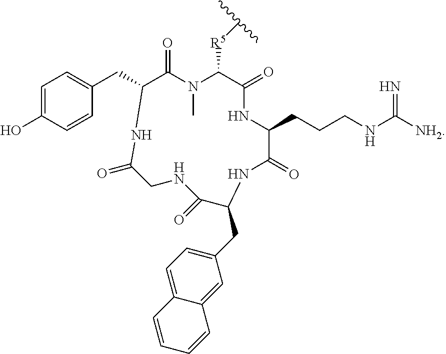

In some embodiments, the polypeptide targeting moiety is a member of a library (e.g., a DNA display library, an RNA display library, a yeast display library, or a phage display library) of polypeptide targeting moieties (e.g., a library including at least 10⁵ polypeptide targeting moieties).

In some embodiments, the binding of the two or more synthetic bifunctional compounds to the target protein is determined using a cell-based assay.

In another aspect, the invention features a method of producing a synthetic bifunctional compound, or a pharmaceutically acceptable salt thereof, that modulates the activity of an extracellular target protein (e.g., a soluble protein, a membrane-bound protein, or a transmembrane protein), wherein the bifunctional compound includes a polypeptide targeting moiety (e.g., an antibody or an antigen binding fragment thereof) that binds to the extracellular target protein covalently conjugated to a small molecule moiety (e.g., a sulfonamide-containing moiety or a hydroxamic acid-containing moiety) that binds to the extracellular target protein, wherein the bifunctional compound binds to the target protein with at least 5-fold greater affinity and/or 5-fold greater selectivity than the affinity of each of the polypeptide targeting moiety and the small molecule moiety alone. The method includes (a) providing (i) a polypeptide targeting moiety; and (ii) a small molecule moiety, wherein at least one of the polypeptide targeting moiety or the small molecule moiety is covalently conjugated to a linker which includes a cross-linking moiety; and (b) reacting the polypeptide targeting moiety and small molecule moiety under conditions sufficient to produce the synthetic bifunctional compound, or a pharmaceutically acceptable salt thereof.

In some embodiments, wherein the small molecule moiety is covalently conjugated to a linker which includes a cross-linking moiety. For example, the method includes reacting (a) a compound having the structure of Formula VII:

A-L-B Formula VII

wherein A includes a small molecule moiety;

L is a linker; and

B is a cross-linking moiety;

with (b) a polypeptide targeting moiety;

under conditions sufficient to result in covalent conjugation between the compound of Formula VII and the polypeptide targeting moiety.

In some embodiments, the cross-linking moiety is a thiol-reactive cross-linking moiety (e.g., a maleimide-containing cross-linking moiety). In some embodiments, the cross-linking moiety is an amine-reactive cross-linking moiety (e.g., a succinimide-containing cross-linking moiety).

In some embodiments, the cross-linking group includes a maleimide, e.g., the cross-linking group includes the structure:

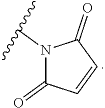 [SMILES: *N1C(=O)C=CC1=O]

In some embodiments, the cross-linking group includes a succinimide, e.g., the cross-linking group includes the structure:

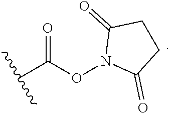 [SMILES: *C(=O)ON1C(=O)CCC1=O]

In some embodiments, the small molecule moiety is covalently conjugated to the polypeptide targeting moiety via a linker (e.g., via a cysteine, a lysine, or a non-natural amino acid in the polypeptide targeting moiety such as a cysteine, lysine, or non-natural amino acid in CDR1, CDR2, or CDR3 of a variable heavy chain, a framework residue within the binding interface, or a framework residue not within the binding interface). In some embodiments, the polypeptide targeting moiety is modified to include a cysteine, lysine, or non-natural amino acid for conjugation to the small molecule moiety. In some embodiments, the small molecule moiety is covalently conjugated to the polypeptide targeting moiety in a site-specific manner. In some embodiments, the linker includes a protein that does not bind to the extracellular target protein (e.g., human aminoguanyltransferase).

In some embodiments, the method further includes reducing the polypeptide targeting moiety under conditions sufficient to reduce at least one disulfide bond prior to reacting the polypeptide targeting moiety with the compound of Formula VII.

In some embodiments, the linker has the structure of Formula I:

A¹-(B¹)_{f}—(C¹)_{g}—(B²)ₕ-(D)-(B³)ᵢ—(C²)ⱼ—(B⁴)ₖ-A² Formula I

wherein A¹ is a bond between the linker and polypeptide targeting moiety; A² is a bond between the small molecule moiety and the linker; B¹, B², B³, and B⁴ each, independently, is selected from optionally substituted C₁-C₂ alkyl, optionally substituted C₁-C₃ heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; f, g, h, I, j, and k are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In another aspect, the disclosure provides a method of producing a synthetic bifunctional compound, or a pharmaceutically acceptable salt thereof, that modulates the activity of an extracellular target protein (e.g., a soluble protein, a membrane-bound protein, or a transmembrane protein). The bifunctional compound includes a polypeptide targeting moiety (e.g., an antibody or an antigen binding fragment thereof) that binds to the extracellular target protein covalently conjugated to a small molecule moiety (e.g., a sulfonamide-containing moiety, a hydroxamic acid-containing moiety, a thiadiazole sulfonamide-containing moiety, a glutamate-urea-lysine-containing moiety, or a cyclic peptide-containing moiety) that binds to the extracellular target protein, wherein the bifunctional compound binds to the target protein with at least 5-fold greater affinity and/or 5-fold greater selectivity than the affinity of each of the polypeptide targeting moiety and the small molecule moiety alone.

In some embodiments, the method includes generating a library of synthetic bifunctional compounds (e.g., by the methods described herein). The library of synthetic bifunctional compounds may be produced, for example, by sitewise diversification (e.g., mutagenesis) of a polypeptide targeting moiety (e.g. a fibronectin type III domain) to produce a diversified library of the polypeptide targeting moiety. The resulting library of the polypeptide targeting moiety may be displayed, for example, on the surface of yeast by yeast display, e.g., as described herein. The library of the polypeptide targeting moiety may be conjugated to the small molecule (e.g., via a linker) by chemical ligation or enzymatic ligation. In some embodiments, the small molecule is conjugated to the polypeptide targeting moiety via a polypeptide linker, and the conjugation of the polypeptide linker to the polypeptide targeting moiety is done by enzymatic ligation.

In some embodiments, the method includes screening a library of synthetic bifunctional compounds for their ability to bind to a target protein (e.g., by the methods described herein).

Chemical Terms

Those skilled in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion, e.g., the interconversion illustrated in the scheme below:

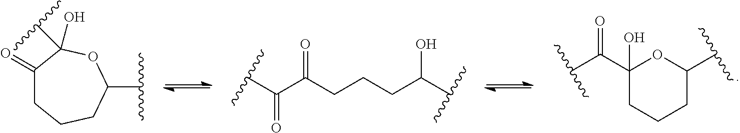 [SMILES: *C1CCCC(=O)C(*)(O)O1 ; *C(=O)C(=O)CCCC(*)O ; *C(=O)C1(O)CCCC(*)O1]

Those skilled in the art will appreciate that, in some embodiments, isotopes of compounds described herein may be prepared and/or utilized in accordance with the present invention. “Isotopes” refers to atoms having the same atomic number but different mass numbers resulting from a different number of neutrons in the nuclei. For example, isotopes of hydrogen include tritium and deuterium. In some embodiments, an isotopic substitution (e.g., substitution of hydrogen with deuterium) may alter the physicochemical properties of the molecules, such as metabolism and/or the rate of racemization of a chiral center.

As is known in the art, many chemical entities (in particular many organic molecules and/or many small molecules) can adopt a variety of different solid forms such as, for example, amorphous forms and/or crystalline forms (e.g., polymorphs, hydrates, solvates, etc). In some embodiments, such entities may be utilized in any form, including in any solid form. In some embodiments, such entities are utilized in a particular form, for example in a particular solid form.

In some embodiments, compounds described and/or depicted herein may be provided and/or utilized in salt form.

In certain embodiments, compounds described and/or depicted herein may be provided and/or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. Furthermore, where a compound includes a plurality of positions at which substitutes are disclosed in groups or in ranges, unless otherwise indicated, the present disclosure is intended to cover individual compounds and groups of compounds (e.g., genera and subgenera) containing each and every individual subcombination of members at each position.

Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The term “alkyl,” as used herein, refers to saturated hydrocarbon groups containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by methyl, ethyl, n- and iso-propyl, n-, sec-, iso- and tert-butyl, neopentyl, and the like, and may be optionally substituted with one, two, three, or, in the case of alkyl groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxyl, optionally substituted with an O-protecting group; (9) nitro; (10) oxo (e.g., carboxyaldehyde or acyl); (11) C₁₋₇ spirocyclyl; (12) thioalkoxy; (13) thiol; (14) —CO₂R^{A′}, optionally substituted with an O-protecting group and where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂(CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alk-C₆₋₁₀ aryl, and (d) hydroxyl; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alk-C₆₋₁₀ aryl; (18) —C(O)R^{D′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alk-C₁₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂(CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂(CH₂CH₂O)ₛ₁ (CH₂)ₛ₃NR^{N1}, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C2-20 alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂o alkyl, (g2) polyethylene glycol of —(CH₂)ₛ₂(OCH₂CH₂)ₛ₁(CH₂)ₛ₃OR′, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂(CH₂CH₂O)ₛ₁ (CH₂)ₛ₃NR^{N1}, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (21) amidine; and (22) silyl groups such as trimethylsilyl, t-butyldimethylsilyl, and tri-isopropylsilyl. In some embodiments, each of these groups can be further substituted as described herein. For example, the alkylene group of a C₁-alkaryl can be further substituted with an oxo group to afford the respective aryloyl substituent.

The term “alkylene” and the prefix “alk-,” as used herein, represent a saturated divalent hydrocarbon group derived from a straight or branched chain saturated hydrocarbon by the removal of two hydrogen atoms, and is exemplified by methylene, ethylene, isopropylene, and the like. The term “C_{x-y} alkylene” and the prefix “C_{x-y} alk-” represent alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁₋₆, C₁₋₁₀, C₂₋₂₀, C₂₋₆, C₂₋₁₀, or C₂₋₂₀ alkylene). In some embodiments, the alkylene can be further substituted with 1, 2, 3, or 4 substituent groups as defined herein for an alkyl group.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the exemplary alkyl substituent groups described herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the exemplary alkyl substituent groups described herein.

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkheterocyclyl (e.g., alkheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the invention can be an unsubstituted amino (i.e., —NH₂) or a substituted amino (i.e., —N(R^{N1})₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₆₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). As used herein, the term “amino acid” in its broadest sense, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide. In some embodiments, the amino acid is attached to the parent molecular group by a carbonyl group, where the side chain or amino group is attached to the carbonyl group. In some embodiments, the amino acid is an α-amino acid. In certain embodiments, the amino acid is a β-amino acid. In some embodiments, the amino acid is a γ-amino acid. Exemplary side chains include an optionally substituted alkyl, aryl, heterocyclyl, alkaryl, alkheterocyclyl, aminoalkyl, carbamoylalkyl, and carboxyalkyl. Exemplary amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxyl; (9) nitro; (10) oxo (e.g., carboxyaldehyde or acyl); (11) C₁₋₇ spirocyclyl; (12) thioalkoxy; (13) thiol; (14) —CO₂R_{A′}, where R_{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₉ aryl, (d) hydrogen, (e) C₁₋₆ alk-C₆₋₁₀ aryl, (f) amino-C₁₋₂o alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₉ alkyl, and (h) amino-polyethylene glycol of —NR^{N1} (CH₂)ₛ₂(CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alk-C₆₋₁₀ aryl; (16) —SO₂R_{D′}, where R_{D′} is selected from the group consisting of (a) C₁₋₆ alkyl, (b) C₆₋₁₉ aryl, (c) C₁₋₆ alk-C₆₋₁₀ aryl, and (d) hydroxyl; (17) —SO₂NR_{E′}R_{F′}, where each of R_{E′} and R_{F′} is, independently, selected from the group consisting of (a) hydrogen, (b) C₁₋₆ alkyl, (c) C₆₋₁₀ aryl and (d) C₁₋₆ alk-C₆₋₁₉ aryl; (18) —C(O)R^{D′}, where R^{G′} is selected from the group consisting of (a) C₁₋₂₉ alkyl (e.g., C₁₋₆ alkyl), (b) C₂₋₂₉ alkenyl (e.g., C₂₋₆ alkenyl), (c) C₆₋₁₉ aryl, (d) hydrogen, (e) C₁₋₆ alk-C₆₋₁₀ aryl, (f) amino-C₁₋₂o alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1} (CH₂)ₛ₂(CH₂CH₂O)ₛ₁ (CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (19) —NR^{H′}C(O)R^{I′}, wherein R^{H′} is selected from the group consisting of (a1) hydrogen and (b1) C₁₋₆ alkyl, and R^{I′} is selected from the group consisting of (a2) C₁₋₂₀ alkyl (e.g., C¹⁻⁶ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂(CH₂CH₂O)ₛ₁ (CH₂)ₛ₃NR^{N1}, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alk-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1} (CH₂)ₛ₂(CH₂CH₂O)ₛ₁ (CH₂)ₛ₃NR^{N1}, wherein S1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

The term “N-alkylated amino acids” as used herein, refers to amino acids containing an optionally substituted C₁ to C₆ alkyl on the nitrogen of the amino acid that forms the peptidic bond. N-alkylated amino acids include, but are not limited to, N-methyl amino acids, such as N-methyl-alanine, N-methyl-threonine, N-methyl-phenylalanine, N-methyl-aspartic acid, N-methyl-valine, N-methyl-leucine, N-methyl-glycine, N-methyl-isoleucine, N(α)-methyl-lysine, N(α)-methyl-asparagine, and N(α)-methyl-glutamine.

The term “aryl,” as used herein, represents a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₁₋₁₀ aryl; (18) —(CH₂)_{q}CONR_{B′}R_{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₁₋₁₀ aryl; (19) —(CH₂)_{q}SO₂R^{D′}, where q is an integer from zero to four and where R^{D′} is selected from the group consisting of (a) alkyl, (b) C₆₋₁₀ aryl, and (c) alk-C₁₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₁₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) C₂₋₂₀ alkenyl; and (27) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “arylalkyl” group, which as used herein, represents an aryl group, as defined herein, attached to the parent molecular group through an alkylene group, as defined herein. Exemplary unsubstituted arylalkyl groups are from 7 to 30 carbons (e.g., from 7 to 16 or from 7 to 20 carbons, such as C₁₋₆ alk-C₆₋₁₀ aryl, C₁₋₁₀ alk-C₆₋₁₀ aryl, or C₁₋₂₀ alk-C₆₋₁₀ aryl). In some embodiments, the alkylene and the aryl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “azido” represents an —N₃ group, which can also be represented as —N═N═N.

The terms “carbocyclic” and “carbocyclyl,” as used herein, refer to an optionally substituted C₃₋₁₂ monocyclic, bicyclic, or tricyclic non-aromatic ring structure in which the rings are formed by carbon atoms. Carbocyclic structures include cycloalkyl, cycloalkenyl, and cycloalkynyl groups.

The “carbocyclylalkyl” group, which as used herein, represents a carbocyclic group, as defined herein, attached to the parent molecular group through an alkylene group, as defined herein. Exemplary unsubstituted carbocyclylalkyl groups are from 7 to 30 carbons (e.g., from 7 to 16 or from 7 to 20 carbons, such as C₁₋₆ alk-C₆₋₁₀ carbocyclyl, carbocyclyl, or C₁₋₂₀ alk-C₆₋₁₀ carbocyclyl). In some embodiments, the alkylene and the carbocyclyl each can be further substituted with 1, 2, 3, or 4 substituent groups as defined herein for the respective groups. Other groups preceded by the prefix “alk-” are defined in the same manner, where “alk” refers to a C₁₋₆ alkylene, unless otherwise noted, and the attached chemical structure is as defined herein.

The term “carbonyl,” as used herein, represents a C(O) group, which can also be represented as C═O.

The term “carboxy,” as used herein, means —CO₂H.

The term “cyano,” as used herein, represents an —CN group.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this invention can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₈ cycloalkyl; (10) C₁₋₆ alk-Cm cycloalkyl; (11) halo; (12) C₁₋₁₂ heterocyclyl (e.g., C₁₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alk-C₆₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “cycloalkylalkyl” group, which as used herein, represents a cycloalkyl group, as defined herein, attached to the parent molecular group through an alkylene group, as defined herein (e.g., an alkylene group of from 1 to 4, from 1 to 6, from 1 to 10, or form 1 to 20 carbons). In some embodiments, the alkylene and the cycloalkyl each can be further substituted with 1, 2, 3, or 4 substituent groups as defined herein for the respective group.

The term “diastereomer,” as used herein means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “halo,” as used herein, represents a halogen selected from bromine, chlorine, iodine, or fluorine.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups. The terms “heteroalkenyl” and heteroalkynyl,” as used herein refer to alkenyl and alkynyl groups, as defined herein, respectively, in which one or two of the constituent carbon atoms have each been replaced by nitrogen, oxygen, or sulfur. In some embodiments, the heteroalkenyl and heteroalkynyl groups can be further substituted with 1, 2, 3, or 4 substituent groups as described herein for alkyl groups.

The term “heteroaryl,” as used herein, represents that subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

The term “heteroarylalkyl” refers to a heteroaryl group, as defined herein, attached to the parent molecular group through an alkylene group, as defined herein. Exemplary unsubstituted heteroarylalkyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heteroaryl, C₁₋₁₀ heteroaryl, or C₁₋₂₀ alk-C₁₋₁₂ heteroaryl). In some embodiments, the alkylene and the heteroaryl each can be further substituted with 1, 2, 3, or 4 substituent groups as defined herein for the respective group. Heteroarylalkyl groups are a subset of heterocyclylalkyl groups.

The term “heterocyclyl,” as used herein represents a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1,2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2,3-dihydro-2-oxo-benzoxazolyl; 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7H-purinyl); 1,2,3,6-tetrahydro-2,6-dioxo-1H purinyl (e.g., 1,2,3,6-tetrahydro-3,7-dimethyl-2,6-dioxo-1H-purinyl); 2-oxobenz[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Heterocyclic groups also include groups of the formula

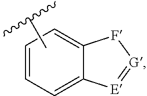

where

E′ is selected from the group consisting of —N— and —CH—; F′ is selected from the group consisting of —N═CH—, —NH—CH₂—, —NH—C(O)—, —NH—, —CH═N—, —CH₂NH—, —C(O)—NH—, —CH═CH—, —CH₂—, —CH₂CH₂—, —CH₂O—, —OCH₂—, —O—, and —S—; and G′ is selected from the group consisting of —CH— and —N—. Any of the heterocyclyl groups mentioned herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alk-C₆₋₁₀ aryl; (8) azido; (9) C₃₋₆ cycloalkyl; (10) C₁₋₆ alk-C₃₋₆ cycloalkyl; (11) halo; (12) C₁₋₁₂ heterocyclyl (e.g., C₂₋₁₂ heteroaryl); (13) (C₁₋₁₂ heterocyclyl)oxy; (14) hydroxyl; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alk-C₆₋₁₀ aryl; (18) —(CH₂)_{q}CONR^{B}R^{C′}, where q is an integer from zero to four and where R^{B′} and R^{C′} are independently selected from the group consisting of (a) hydrogen, (b) C₁₋₆ alkyl, (c) C₆₋₁₀ aryl, and (d) C₁₋₆ alk-C₆₋₁₀ aryl; (19) —(CH₂)_{q}SO₂R^{D′}, where q is an integer from zero to four and where R^{D′} is selected from the group consisting of (a) C₁₋₆ alkyl, (b) C₆₋₁₀ aryl, and (c) C₁₋₆ alk-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₁₋₆ alkyl, (c) C₆₋₁₀ aryl, and (d) C₁₋₆ alk-C₆₋₁₀ aryl; (21) thiol; (22) C₆₋₁₀ aryloxy; (23) C₃₋₆ cycloalkoxy; (24) arylalkoxy; (25) C₁₋₆ alk-C₁₋₁₂ heterocyclyl (e.g., C₁₋₆ alk-C₁₋₁₂ heteroaryl); (26) oxo; (27) (C₁₋₁₂ heterocyclyl)imino; (28) C₂₋₂₀ alkenyl; and (29) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl)oyl substituent group.

The “heterocyclylalkyl” group, which as used herein, represents a heterocyclyl group, as defined herein, attached to the parent molecular group through an alkylene group, as defined herein. Exemplary unsubstituted heterocyclylalkyl groups are from 2 to 32 carbons (e.g., from 2 to 22, from 2 to 18, from 2 to 17, from 2 to 16, from 3 to 15, from 2 to 14, from 2 to 13, or from 2 to 12 carbons, such as C₁₋₆ alk-C₁₋₁₂ heterocyclyl, alk-C₁₋₁₂ heterocyclyl, or C₁₋₂₀ alk-C₁₋₁₂ heterocyclyl). In some embodiments, the alkylene and the heterocyclyl each can be further substituted with 1, 2, 3, or 4 substituent groups as defined herein for the respective group.

The term “hydrocarbon,” as used herein, represents a group consisting only of carbon and hydrogen atoms.

The term “hydroxyl,” as used herein, represents an —OH group. In some embodiments, the hydroxyl group can be substituted with 1, 2, 3, or 4 substituent groups (e.g., O-protecting groups) as defined herein for an alkyl.

The term “isomer,” as used herein, means any tautomer, stereoisomer, enantiomer, or diastereomer of any compound of the invention. It is recognized that the compounds of the invention can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “N-protected amino,” as used herein, refers to an amino group, as defined herein, to which is attached one or two N-protecting groups, as defined herein.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3^{rd} Edition (John Wiley & Sons, New York, 1999), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups, such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “O-protecting group,” as used herein, represents those groups intended to protect an oxygen containing (e.g., phenol, hydroxyl, or carbonyl) group against undesirable reactions during synthetic procedures. Commonly used O-protecting groups are disclosed in Greene, “Protective Groups in Organic Synthesis,” 3^{rd} Edition (John Wiley & Sons, New York, 1999), which is incorporated herein by reference. Exemplary O-protecting groups include acyl, aryloyl, or carbamyl groups, such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl; alkylcarbonyl groups, such as acyl, acetyl, propionyl, pivaloyl, and the like; optionally substituted arylcarbonyl groups, such as benzoyl; silyl groups, such as trimethylsilyl (TMS), tert-butyldimethylsilyl (TBDMS), tri-iso-propylsilyloxymethyl (TOM), triisopropylsilyl (TIPS), and the like; ether-forming groups with the hydroxyl, such methyl, methoxymethyl, tetrahydropyranyl, benzyl, p-methoxybenzyl, trityl, and the like; alkoxycarbonyls, such as methoxycarbonyl, ethoxycarbonyl, isopropoxycarbonyl, n-isopropoxycarbonyl, n-butyloxycarbonyl, isobutyloxycarbonyl, sec-butyloxycarbonyl, t-butyloxycarbonyl, 2-ethylhexyloxycarbonyl, cyclohexyloxycarbonyl, methyloxycarbonyl, and the like; alkoxyalkoxycarbonyl groups, such as methoxymethoxycarbonyl, ethoxymethoxycarbonyl, 2-methoxyethoxycarbonyl, 2-ethoxyethoxycarbonyl, 2-butoxyethoxycarbonyl, 2-methoxyethoxymethoxycarbonyl, allyloxycarbonyl, propargyloxycarbonyl, 2-butenoxycarbonyl, 3-methyl-2-butenoxycarbonyl, and the like; haloalkoxycarbonyls, such as 2-chloroethoxycarbonyl, 2-chloroethoxycarbonyl, 2,2,2-trichloroethoxycarbonyl, and the like; optionally substituted arylalkoxycarbonyl groups, such as benzyloxycarbonyl, p-methylbenzyloxycarbonyl, p-methoxybenzyloxycarbonyl, p-nitrobenzyloxycarbonyl, 2,4-dinitrobenzyloxycarbonyl, 3,5-dimethylbenzyloxycarbonyl, p-chlorobenzyloxycarbonyl, p-bromobenzyloxy-carbonyl, fluorenylmethyloxycarbonyl, and the like; and optionally substituted aryloxycarbonyl groups, such as phenoxycarbonyl, p-nitrophenoxycarbonyl, o-nitrophenoxycarbonyl, 2,4-dinitrophenoxycarbonyl, p-methylphenoxycarbonyl, m-methylphenoxycarbonyl, o-bromophenoxycarbonyl, 3,5-dimethylphenoxycarbonyl, p-chlorophenoxycarbonyl, 2-chloro-4-nitrophenoxy-carbonyl, and the like); substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl); carbonyl-protecting groups (e.g., acetal and ketal groups, such as dimethyl acetal, 1,3-dioxolane, and the like; acylal groups; and dithiane groups, such as 1,3-dithianes, 1,3-dithiolane, and the like); carboxylic acid-protecting groups (e.g., ester groups, such as methyl ester, benzyl ester, t-butyl ester, orthoesters, and the like; and oxazoline groups.

The term “oxo” as used herein, represents ═O.

The prefix “perfluoro,” as used herein, represents anyl group, as defined herein, where each hydrogen radical bound to the alkyl group has been replaced by a fluoride radical. For example, perfluoroalkyl groups are exemplified by trifluoromethyl, pentafluoroethyl, and the like.

The term “protected hydroxyl,” as used herein, refers to an oxygen atom bound to an O-protecting group.

The term “spirocyclyl,” as used herein, represents a C₂₋₇ alkylene diradical, both ends of which are bonded to the same carbon atom of the parent group to form a spirocyclic group, and also a C₁₋₆ heteroalkylene diradical, both ends of which are bonded to the same atom. The heteroalkylene radical forming the spirocyclyl group can containing one, two, three, or four heteroatoms independently selected from the group consisting of nitrogen, oxygen, and sulfur. In some embodiments, the spirocyclyl group includes one to seven carbons, excluding the carbon atom to which the diradical is attached. The spirocyclyl groups of the invention may be optionally substituted with 1, 2, 3, or 4 substituents provided herein as optional substituents for cycloalkyl and/or heterocyclyl groups.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

The term “sulfonyl,” as used herein, represents an —S(O)₂— group.

The term “thiol,” as used herein, represents an —SH group.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “active site” refers to the location on a protein (e.g., an enzyme) where substrate molecules bind and undergo a chemical reaction. By “does not bind at the active site” is meant that no atoms of a moiety substantially participate in binding with residues within the active site (e.g., residues that participate in binding to a natural substrate molecule).

As is known in the art, “affinity” is a measure of the tightness with which a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

As used herein, the term “antagonist” refers to a compound that i) partially of fully inhibits, decreases or reduces the effects of a target protein (e.g., a eukaryotic target protein such as a mammalian target protein or a fungal target protein or a prokaryotic target protein such as a bacterial target protein); and/or ii) partially or fully inhibits, decreases, reduces, or delays one or more biological events. An antagonist may be direct (in which case it exerts its influence directly upon its target) or indirect (in which case it exerts its influence by other than binding to its target; e.g., by interacting with a regulator of the target protein (e.g., a eukaryotic target protein such as a mammalian target protein or a fungal target protein or a prokaryotic target protein such as a bacterial target protein), for example so that level or activity of the target protein is altered). In some embodiments, an antagonist is an inverse agonists (e.g., a compound that prevents activation of a target protein by an agonist).

As used herein, the term “agonist” refers to a compound that i) activates or increases (e.g., increases the rate of onset or occurrence) the effects of a target protein (e.g., a eukaryotic target protein or a fungal target protein or a prokaryotic target protein such as a bacterial target protein); and/or ii) activates or increases (e.g., increases the rate of onset or occurrence) one or more biological events. An agonist may be direct (in which case it exerts its influence directly upon its target) or indirect (in which case it exerts its influence by other than binding to its target; e.g., by interacting with a regulator of the target protein (e.g., a eukaryotic target protein such as a mammalian target protein or a fungal target protein or a prokaryotic target protein such as a bacterial target protein), for example so that the level or activity of the target protein is altered). The term agonist is also considered to include a “partial agonist,” which refers to a compound that activates or increases the effects of a target protein and/or activates or increases one or more biological events, but which only has partial efficacy relative to a full agonist. In some embodiments, the target protein is a protein that is displayed (e.g., transiently displayed) on the extracellular surface of a cell (e.g., in response to a cell stimulus, such as the activation of a cell signaling pathway). For example, the target protein may be an intracellular protein that is secreted to the surface of a cell in response to a cell stimulus, e.g., an intracellular protein that is secreted to the surface of a tumor cell. Accordingly, for the purposes of this invention, an intracellular protein (e.g., a soluble intracellular protein) which is at any time displayed on the surface of a cell (e.g., as a membrane-bound protein), may also be considered to be an extracellular target protein.

As used herein, “antibody” refers to a polypeptide whose amino acid sequence including immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

As used herein, “antigen-binding fragment” refers to a portion of an antibody that retains the binding characteristics of the parent antibody.

As used herein, the terms “approximately” and “about” are each intended to encompass normal statistical variation as would be understood by those of ordinary skill in the art as appropriate to the relevant context. In certain embodiments, the terms “approximately” or “about” each refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

It will be understood that the term “binding” as used herein, typically refers to association (e.g., non-covalent or covalent) between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

The affinity of a molecule X for its partner Y can generally be represented by the dissociation constant (K_{D}). Affinity can be measured by common methods known in the art, including those described herein. Specific illustrative and exemplary embodiments for measuring binding affinity are described below. The term “K_{D},” as used herein, is intended to refer to the dissociation equilibrium constant of a particular compound-protein interaction. Typically, the compounds of the invention bind to target proteins (e.g., a eukaryotic target protein such as a mammalian target protein or a fungal target protein or a prokaryotic target protein such as a bacterial target protein) with a dissociation equilibrium constant (K_{D}) of less than about 10⁻⁶ M, such as less than approximately 10⁻⁷ M, 10⁻⁸ M, 10⁻⁹M, or 10⁻¹⁹ M or even lower or between 10⁻⁷M and 10⁻⁸ M, between 10⁻⁸ M and 10⁻⁹ M, or between 10⁻⁹ M and 10⁻¹⁹ M, e.g., when determined by surface plasmon resonance (SPR) technology using the target protein as the analyte and the compound as the ligand.

As used herein, the term “conjugate” refers to a compound formed by the joining (e.g., via a covalent bond forming reaction) of two or more chemical compounds (e.g., a compound including a small molecule moiety and a polypeptide targeting moiety).

As used herein, the term “cross-linking group” refers to a group comprising a reactive functional group capable of chemically attaching to specific functional groups (e.g., primary amines, sulfhydryls) on proteins or other molecules.

Many methodologies described herein include a step of “determining.” Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

As used herein, the term “free cysteine” refers to a cysteine residue present in a biologic, whether on-diagonal or off-diagonal, that is not involved in a disulfide bond.

The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate compound that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, a modulator is an allosteric modulator such as an allosteric agonist.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, refers to those salts of the compounds described here that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

As used herein, the term “reactive amino acid residue” refers to a natural or non-natural amino acid comprising a functional group (e.g., a nucleophilic functional group) capable of chemically attaching to specific functional groups (e.g., a cross-linking group). Examples of reactive amino acids include cysteine, lysine, serine, and amino acids having azides on the side chain. “Non-reactive amino acids” refers to natural or non-natural amino acids that do not contain a functional group capable of chemically attaching to specific functional groups. Examples of non-reactive amino acids include valine, alanine, isoleucine, theronine, and leucine.

The term “reference” is often used herein to describe a standard or control compound, individual, population, sample, sequence or value against which a compound, individual, population, sample, sequence or value of interest is compared. In some embodiments, a reference compound, individual, population, sample, sequence or value is tested and/or determined substantially simultaneously with the testing or determination of the compound, individual, population, sample, sequence or value of interest. In some embodiments, a reference compound, individual, population, sample, sequence or value is a historical reference, optionally embodied in a tangible medium. Typically, as would be understood by those skilled in the art, a reference compound, individual, population, sample, sequence or value is determined or characterized under conditions comparable to those utilized to determine or characterize the compound, individual, population, sample, sequence or value of interest.

The term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating compound. In some embodiments, a small molecule is biologically active. In some embodiments, a small molecule is detectable (e.g., comprises at least one detectable moiety). In some embodiments, a small molecule is a therapeutic.

Those of ordinary skill in the art, reading the present disclosure, will appreciate that certain small molecule compounds described herein may be provided and/or utilized in any of a variety of forms such as, for example, salt forms, protected forms, pro-drug forms, ester forms, isomeric forms (e.g., optical and/or structural isomers), isotopic forms, etc. In some embodiments, reference to a particular compound may relate to a specific form of that compound. In some embodiments, reference to a particular compound may relate to that compound in any form. In some embodiments, where a compound is one that exists or is found in nature, that compound may be provided and/or utilized in accordance in the present invention in a form different from that in which it exists or is found in nature. Those of ordinary skill in the art will appreciate that a compound preparation including a different level, amount, or ratio of one or more individual forms than a reference preparation or source (e.g., a natural source) of the compound may be considered to be a different form of the compound as described herein. Thus, in some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form; etc.

As used herein, the term “small molecule binding site” refers to a location on a protein (e.g., a GPCR, an ion channel, or an enzyme) where a small molecule is capable of binding. For example, small molecule binding sites include, but are not limited to, active sites of proteins which bind natural substrates and allosteric sites on proteins which may or may not bind a natural substrate, and are distinct from the active site.

As used herein, the terms “specific binding” or “specific for” or “specific to” refer to an interaction between a binding agent and a target entity. As will be understood by those of ordinary skill, an interaction is considered to be “specific” if it is favored in the presence of alternative interactions, for example, binding with a K_{D} of less than 10 μM (e.g., less than 5 μM, less than 1 μM, less than 500 nM, less than 200 nM, less than 100 nM, less than 75 nM, less than 50 nM, less than 25 nM, less than 10 nM, between 5 μM and 1 μM, between 1 μM and 500 nM, between 500 nM and 200 nM, between 200 nM and 100 nM, between 100 nM and 75 nM, between 75 nM and 50 nM, between 50 nM and 25 nM, between 25 nM and 10 nM). In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent.

The term “specific” when used with reference to a compound having an activity, is understood by those skilled in the art to mean that the compound discriminates between potential target entities or states. For example, in some embodiments, a compound is said to bind “specifically” to its target if it binds preferentially with that target in the presence of one or more competing alternative targets. In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “target protein” refers to any protein that participates in a biological pathway associated with a disease, disorder or condition. In some embodiments, a target protein is a naturally-occurring protein; in some such embodiments, a target protein is naturally found in certain mammalian cells (e.g., a mammalian target protein), fungal cells (e.g., a fungal target protein), bacterial cells (e.g., a bacterial target protein) or plant cells (e.g., a plant target protein). Target proteins can be naturally occurring, e.g., wild type. Alternatively, the target protein can vary from the wild type protein but still retain biological function, e.g., as an allelic variant, a splice mutant or a biologically active fragment. In some embodiments, the target protein is an extracellular target protein. Extracellular target proteins of the invention are considered to include soluble proteins, membrane-bound proteins, and/or transmembrane proteins. A soluble protein may be considered to include any polypeptide (e.g., a globular protein or globular protein domain) that is soluble in an aqueous medium (e.g., the cytoplasm, the extracellular fluid, lymph, or blood). A membrane-bound protein is considered to include any protein that is associated (e.g., by non-covalent interaction or by covalent linkage) with a cellular membrane. Membrane-bound proteins may also be soluble proteins that interact transiently with a membrane. Transmembrane proteins are considered to include any polypeptide that spans (e.g., partially spans or fully spans) a cellular membrane. Transmembrane proteins may include an extracellular domain, transmembrane domain, and/or an intracellular domain. In some embodiments, the target protein is the extracellular domain of a transmembrane protein.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “wild-type” refers to an entity having a structure and/or activity as found in nature in a “normal” (as contrasted with mutant, diseased, altered, etc) state or context. Those of ordinary skill in the art will appreciate that wild-type genes and polypeptides often exist in multiple different forms (e.g., alleles).

As used herein, the term “solvent exposed amino acid” refers to an amino acid that is accessible to the solvent surrounding the protein. In some embodiments, a solvent exposed amino acid is an amino acid that when substituted does not substantially change the three-dimensional structure of the protein.

### BRIEF DESCRIPTION OF THE DRAWINGS

FIG. 1A-B is diagram depicting bifunctional compounds of the invention bound to a polypeptide. FIG. 1A shows a bifunctional compound bound to a polypeptide in which the polypeptide targeting moiety and the small molecule bind to the polypeptide, at least in part, at the same site. FIG. 1B shows a bifunctional compound bound to a polypeptide in which the polypeptide targeting moiety and the small molecule bind to the polypeptide at distinct sites on the polypeptide.

FIG. 2A-B is a diagram and corresponding table showing the structure and solvent exposed amino acid sites of a wild-type tenth fibronectin type III domain (Fn). FIG. 2A is diagram showing the NMR structure of a wild-type tenth fibronectin type III domain, solved via NMR. Frequently mutated sites (e.g., the BC, DE, and FG loops) are located on three loops (denoted by boxes). Residue T28 (represented as a ball) is a solvent exposed amino acid located on one of the frequently diversified regions, and may be used as a site for conjugation of a linker. FIG. 2B is a table showing solvent-accessible surface area (SASA) for sites within the frequently diversified regions.

FIG. 3A-C is a series of graphs showing that maleimide-fluorescein is effectively conjugated to yeast-displayed Fn with a single cysteine. FIG. 3A is a trace generated by fluorescence-activated cell sorting (FACS) analysis of yeast displaying either the unlabeled yeast, the cysteine-free parental Fn domain (T28), or the cysteine mutant Fn domain (T28C). FIG. 3B is a graph showing the concentration-dependent conjugation of maleimide-fluorescein to Fn-T28C. FIG. 3C is a graph showing the time-dependent conjugation of maleiminde-fluorescein to Fn-T28C.

FIG. 4 is a FACS trace showing that maleimide-acetazolamide (AAZ) is effectively conjugated to yeast-displayed Fn. Conjugation with maleimide-AAZ reduced fluorescein conjugation by 70%, which is consistent with effective AAZ conjugation.

FIG. 5 is FACS trace showing that yeast-displayed Fn-AAZ binds carbonic anhydrase 9.

FIG. 6A-B is graph and corresponding set of images showing that yeast-displayed Fn-AAZ is functional at multiple Fn conjugation sites. FIG. 6A is a graph showing the relative binding of Fn-AAZ to carbonic anhydrase 9, wherein in the AAZ is conjugated to Fn through various solvent exposed amino acids (D80, R78, R30, or T28) and using various PEG linkers of different lengths (PEG2, PEG3, PEG5, or PEG7). FIG. 6B is two images depicting the location of the solvent exposed amino acids used for conjugation to Fn mapped onto the structure of Fn (side-view and top view).

FIG. 7 is a diagram showing the design for the construction of combinatorial libraries of yeast-displayed Fn.

FIG. 8 is series of FACS traces showing that Fn and AAZ provide a mutual benefit in binding to carbonic anhydrase 9. Binding is enabled by the combination of select Fn clones and the AAZ conjugation, with longer PEG lengths enabling greater binding.

FIG. 9 is a series of FACS traces showing that sub-libraries of yeast-displayed Fn-AAZ conjugates can be identified (boxes in upper right quandrant) which bind with high affinity to carbonic anhydrase 9.

FIG. 10A-B is a series of FACS traces showing that yeast-displayed Fn-AAZ clones bind selectively to carbonic anhydrase 9 over carbonic anhydrase 2. FIG. 10A shows that Fn-AAZ clones conjugated via Cys28 are selective for carbonic anhydrase 9 over carbonic anhydrase 2. FIG. 10B shows that Fn-AAZ clones conjugated via Cys80 are selective for carbonic anhydrase 9 over carbonic anhydrase 2.

FIG. 11 is a graph showing that, in the absence of an Fn domain, AAZ-Fluorescein is not highly selective for carbonic anhydrase 9 (K_{d}=340 nM) over and carbonic anhydrase 2 (K_{d}=560 nM).

FIG. 12 is a series of FACS traces showing that libraries of yeast-displayed Fn-AAZ conjugates can be enriched to identify clones that bind with high affinity to carbonic anhydrase 2.

FIG. 13A-C is a series of images that shows the expression and purification of an individual Fn-AAZ clone. FIG. 13A is a polyacrylamide gel showing the expression of an Fn-AAZ clone. FIG. 13B is a mass spectrometry trace showing the purified Fn clone (M=11835 Da). FIG. 13C is a mass spectrometry trace of the purified Fn clone after maleimide conjugation to PEG₅AAZ (M=12374 Da).

FIG. 14A-B is a series of graphs showing the binding characteristics of each of two exemplary purified clones of Fn-AAZ to carbonic anhydrase 9. FIG. 14A shows that a purified Fn-AAZ clone having the amino acid sequence of SEQ ID NO. 3 (Clone 0.3.10) binds with 10-fold higher affinity to carbonic anhydrase 9 when compared to Fn that has not been conjugated to AAZ. FIG. 14B shows that a purified Fn-AAZ clone having the amino acid sequence of SEQ ID NO. 4 (Clone 0.3.9) binds with approximately 6-fold higher affinity to carbonic anhydrase 9 when compared to Fn that has not been conjugated to AAZ. FIG. 15 is a graph showing the % yield of yeast recovered following two rounds of FACS sorting of a yeast-displayed library of Fn domains conjugated to a cyclic peptide (CP). FACS sorting was performed to identify Fn-CP clones that bind to the polypeptide target, CXCR4.

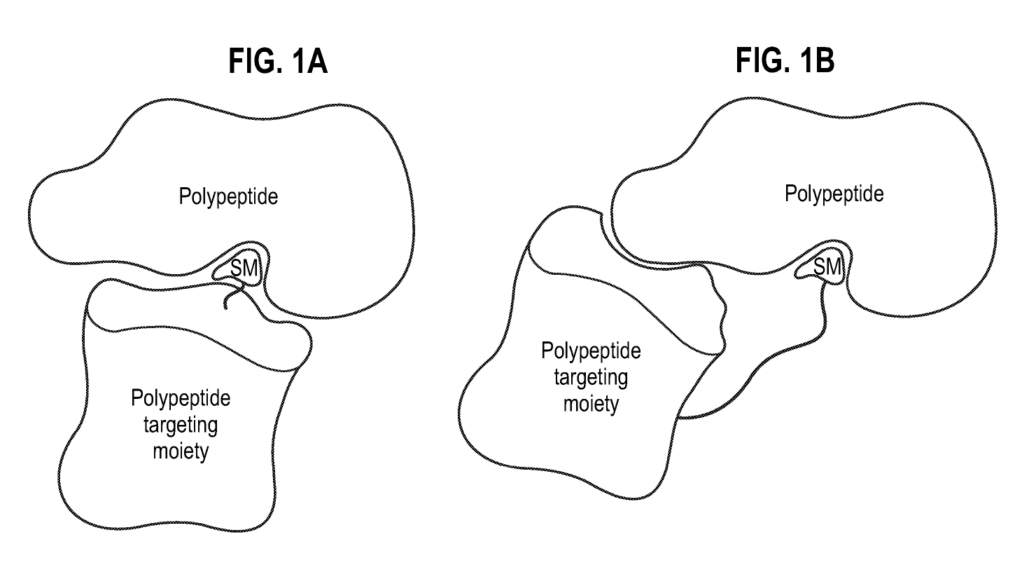

### DETAILED DESCRIPTION

### Small Molecule Moieties

The small molecule moieties of the invention include low molecular weight organic and/or inorganic compounds which have affinity (e.g., low affinity) for the target protein when not covalently conjugated to the polypeptide targeting moiety. In some embodiments, the small molecule moiety binds at the active site of the target protein. In some embodiments, the small molecule moiety does not specifically bind to the target protein (e.g., the small molecule binds to more than one member of the family of proteins to which the target protein belongs.)

In some embodiments, a small molecule is an antagonist (e.g., a direct inhibitor) of the target protein. In some embodiments, the small molecule is an agonist or a partial agonist (e.g., a direct agonist of partial agonist) of the target protein.

In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating compound. In some embodiments, a small molecule is biologically active. In some embodiments, a small molecule is detectable (e.g., comprises at least one detectable moiety). In some embodiments, a small molecule is a therapeutic.

In some embodiments, the small molecule moiety is a sulfonamide. For example, the small molecule moiety includes the structure of Formula II:

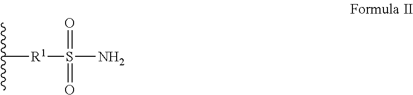

wherein R¹ is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the small molecule moiety is a hydroxamic acid. For example, the small molecule moiety includes the structure of Formula III:

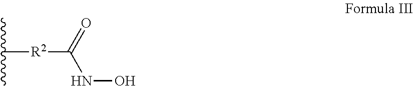

wherein R² is optionally substituted C₁-C₆ alkyl, optionally substituted C₁-C₆ heteroalkyl, optionally substituted C₆-C₁₀ aryl, or optionally substituted C₂-C₉ heteroaryl.

In some embodiments, the small molecule moiety is a thiadiazole sulfonamide or includes a glutamate-urea-lysine moiety. For example, the small molecule moiety includes the structure of Formula IV or Formula V:

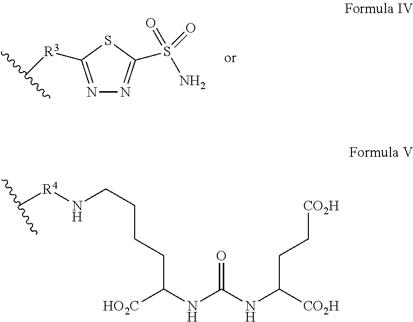

wherein R³ is optionally substituted C₁-C₆ alkyl or optionally substituted C₁-C₆ heteroalkyl (e.g., —NHC(O)—); and

R⁴ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

In some embodiments, the small molecule moiety includes the structure:

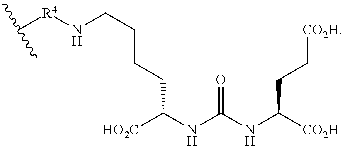

In some embodiments, the small molecule moiety is a cyclic peptide. For example, the small molecule moiety includes the structure of Formula VI:

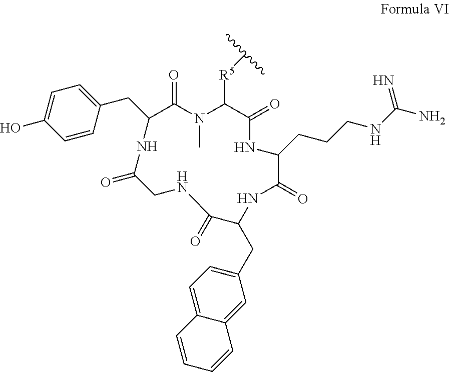

wherein R⁵ is optionally substituted C₁-C₆ alkyl, optionally substituted C₁-C₆ heteroalkyl, optionally substituted C₆-C₁₀ aryl, optionally substituted C₂-C₆ heteroaryl, or optionally substituted C₆-C₁₀ aryl C₁-C₆ alkyl, or optionally substituted C₂-C₆ heteroaryl C₁-C₆ alkyl.

In some embodiments, the small molecule moiety includes the structure:

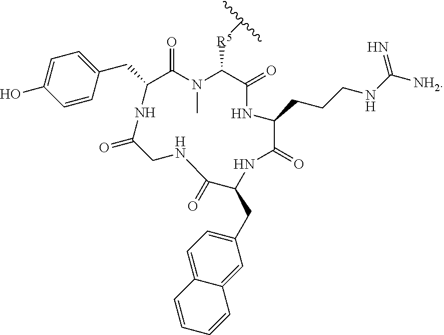

### Linkers

The compounds of the invention include a linker (e.g., moiety linker joining a small molecule moiety to a polypeptide targeting moiety). The linker component of the invention is, at its simplest, a bond, but may also provide a linear, cyclic, or branched molecular skeleton having pendant groups covalently linking two moieties.

Thus, a linker, when included in a compound and/or conjugate as described herein, achieves linking of two (or more) moieties by covalent means, involving bond formation with one or more functional groups located on either moiety. Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

In some embodiments, such covalent linking of two (or more) moieties may be effected using a linker that contains reactive moieties capable of reaction with such functional groups present in either moiety. For example, an amine group of a moiety may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:
(i) α-haloacetyl compounds, which show specificity towards amino groups in the absence of reactive thiol groups and are of the type XCH₂CO— (where X═Br, Cl, or I), for example, as described by Wong Biochemistry 24:5337 (1979);
(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);
(iii) aryl halides such as reactive nitrohaloaromatic compounds;
(iv) alkyl halides, as described, for example, by McKenzie et al., J. Protein Chem. 7:581 (1988);
(v) aldehydes and ketones capable of Schiff's base formation with amino groups, the adducts formed usually being stabilized through reduction to give a stable amine;
(vi) epoxide derivatives such as epichlorohydrin and bisoxiranes, which may react with amino, sulfhydryl, or phenolic hydroxyl groups;
(vii) chlorine-containing derivatives of s-triazines, which are very reactive towards nucleophiles such as amino, sufhydryl, and hydroxyl groups;
(vii) aziridines based on s-triazine compounds detailed above, e.g., as described by Ross, J. Adv. Cancer Res. 2:1 (1954), which react with nucleophiles such as amino groups by ring opening;
(ix) squaric acid diethyl esters as described by Tietze, Chem. Ber. 124:1215 (1991); and
(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:
(i) isocyanates and isothiocyanates, particularly aromatic derivatives, which form stable urea and thiourea derivatives respectively;
(ii) sulfonyl chlorides, which have been described by Herzig et al., Biopolymers 2:349 (1964);
(iii) acid halides;
(iv) active esters such as nitrophenylesters or N-hydroxysuccinimidyl esters;
(v) acid anhydrides such as mixed, symmetrical, or N-carboxyanhydrides;
(vi) other useful reagents for amide bond formation, for example, as described by M. Bodansky, Principles of Peptide Synthesis, Springer-Verlag, 1984;
(vii) acylazides, e.g., wherein the azide group is generated from a preformed hydrazide derivative using sodium nitrite, as described by Wetz et al., Anal. Biochem. 58:347 (1974);
(vii) imidoesters, which form stable amidines on reaction with amino groups, for example, as described by Hunter and Ludwig, J. Am. Chem. Soc. 84:3491 (1962); and
(ix) haloheteroaryl groups such as halopyridine or halopyrimidine.

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in either moiety may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of one moiety with a reactive chemical group of the other without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within either moiety, resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between the two moieties.

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₂-C₁₀₀ polyethylene glycol, or C₁₋₁₀ heteroalkyl.

In some embodiments, the spacer element of a linker consists of a polyethylene glycol. Polyethylene glycols of the invention are considered to include an alkoxy chain comprised of one or more momomer units, each monomer unit consisting of —OCH2CH2-. Polyethyelene glycol (PEG) is also sometimes referred to as polyethylene oxide (PEO) or polyoxyethylene (POE), and these terms may be considered interchangeable for the purpose of this invention. For example, a polyethylene glycol may have the structure, —(CH2)s2(OCH2CH2)s1(CH2)s3O—, wherein s1 is an integer from 1 to 10 (e.g., from 1 to 6 or from 1 to 4), and each of s2 and s3, independently, is an integer from 0 to 10 (e.g., from 0 to 4, from 0 to 6, from 1 to 4, from 1 to 6, or from 1 to 10). In some embodiments, the PEG is PEG₂, PEG₃, PEG₅, or PEG₇, wherein the subscript refers to the number of —OCH2CH2-monomer units of the PEG. Polyethylene glycol may also be considered to include an amino-polyethylene glycol of —NRN1(CH2)s2(CH2CH2O)s1(CH2)s3NRN1-, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each RN1 is, independently, hydrogen or optionally substituted C1-6 alkyl.

In some embodiments, the linker includes a polypeptide having two or more amino acid residues. In some embodiments, the linker is a polypeptide having the formula (G₄S)ₙ, where n is 1 or greater (e.g., n is 2, 3, 4, 5, 6, 7, 8, 9, 10, 11, 12, 13, 14, 15, 16, 17, 18, 19, 20). In some embodiments, the linker is a polypeptide having the formula (G₄S)₅. In some embodiments, the linker is a polypeptide having the formula (G₄S)₁₀. In some embodiments, the linker is a polypeptide having the formula (G₄S)₁₅. In some embodiments, the linker is a polypeptide having the formula (G₄S)ₙ(EAK)ₘ, where n and m each independently have a value or 1 or greater (e.g., n is 2, 3, 4, 5, 6, 7, 8, 9, 10, 11, 12, 13, 14, 15, 16, 17, 18, 19, 20). In some embodiments, the linker is a polypeptide having the formula (G₄S)₅(EAK)₅. In some embodiments, the linker is a polypeptide having the formula (G₄S)₁₀(EAK)₅. In some embodiments, the linker is a polypeptide having the formula (G₄S)ₙ(EAK)ₘ(G₄S)ₖ, where n, m, and k each independently have a value or 1 or greater (e.g., n is 2, 3, 4, 5, 6, 7, 8, 9, 10, 11, 12, 13, 14, 15, 16, 17, 18, 19, 20). In some embodiments, the linker is a polypeptide having the formula (G₄S)₅(EAK)₁₀(G₄S)₅.

In some instances, the linker is described by Formula V.

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

In some embodiments, the linker is a bond or a linear chain of up to 10 atoms, independently selected from carbon, nitrogen, oxygen, sulfur or phosphorous atoms, wherein each atom in the chain is optionally substituted with one or more substituents independently selected from alkyl, alkenyl, alkynyl, aryl, heteroaryl, chloro, iodo, bromo, fluoro, hydroxyl, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and wherein any two atoms in the chain may be taken together with the substituents bound thereto to form a ring, wherein the ring may be further substituted and/or fused to one or more optionally substituted carbocyclic, heterocyclic, aryl, or heteroaryl rings. In some embodiments, a linker has the structure of Formula I:

A¹-(B¹)ₐ—(C¹)_{b}—(B²)_{c}-(D)-(B³)_{d}—(C²)ₑ—(B⁴)_{f}-A² Formula I

where A¹ is a bond between the linker and polypeptide targeting moiety; A² is a bond between the small molecule moiety and the linker; B¹, B², B³, and B⁴ each, independently, is selected from optionally substituted C₁-C₂ alkyl, optionally substituted C₁-C₃ heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; a, b, c, d, e, and f are each, independently, 0 or 1; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)ₐ—(C¹)_{b}—(B²)_{c}— to —(B³)_{d}—(C²)ₑ—(B⁴)_{f}- A².

### Polypeptides

Polypeptides include, for example, any of a variety of hematologic agents (including, for instance, erythropoietin, blood-clotting factors, etc.), interferons, colony stimulating factors, antibodies, enzymes, and hormones. The identity of a particular polypeptide is not intended to limit the present disclosure, and any polypeptide of interest can be a polypeptide in the present methods.

In some embodiments, the polypeptide is an ion channel, such as a leukocyte ion channel (e.g., CRAC or Kv1.3), a TRP channel (e.g., TRPV1), a purinergic receptor (e.g., a P2X or P2Y receptor), or an epithelial sodium channel (ENaC).

In some embodiments, the polypeptide is a GPCR, such as an adenosine receptor (AR), an endothelin receptor (ETR), a bradykinin receptor (BKR), an angiotensin receptor (AIR or AIIR), a cannabinoid receptor (CNR), a muscarinic receptor, a neurotensin receptor (NTR), a C5a receptor (C5aR), a purinergic receptor (e.g., a P2X or P2Y receptor), calcitonin gene-related peptide receptor (CGRP-R), or glucagon-like peptide 1 receptor (GLP1R).

In some embodiments, the polypeptide is an enzyme, such an enzyme of the PCSK family (e.g., furin), matriptase, prostasin, MT1-MMP (also called MMP14), a disintegrin and metalloproteinase (ADAMS), Factor Xia, Factor D, transglutaminases 2, cathepsin S, CD73, CD39, or membrane guanylyl cyclase C.

### Polypeptide Targeting Moieties

A reference polypeptide described herein can include a target-binding domain that binds to a target of interest (e.g., binds to an antigen). For example, a polypeptide, such as an antibody, can bind to a transmembrane polypeptide (e.g., receptor) or ligand (e.g., a growth factor). In some embodiments, the extracellular target proteins of the present invention include proteins which have a small molecule binding site (e.g., an active site). Exemplary molecular targets (e.g., antigens) for polypeptides described herein (e.g., antibodies) include GPCRs (e.g., Class A GPCRs such as CXCR4, Class B GPCRs, Class C GPCRs, Class D GPCRs, Class E GPCRs and Class F GPCRs), ion channels (e.g., Trp channels, Nav 1.7 channels, and CRAC channels), and enzymes (e.g., carbonic anhydrases and metalloproteases).

Antibodies

An IgG antibody consists of two identical light polypeptide chains and two identical heavy polypeptide chains linked together by disulfide bonds. The first domain located at the amino terminus of each chain is variable in amino acid sequence, providing the antibody binding specificities found in each individual antibody. These are known as variable heavy (VH) and variable light (VL) regions. The other domains of each chain are relatively invariant in amino acid sequence and are known as constant heavy (CH) and constant light (CL) regions. For an IgG antibody, the light chain includes one variable region (VL) and one constant region (CL). An IgG heavy chain includes a variable region (VH), a first constant region (CH1), a hinge region, a second constant region (CH2), and a third constant region (CH3). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)₂ fragment, a Fd fragment, a Fv fragment, a scFv fragment, a dAb fragment (Ward et al., (1989) Nature 341:544-546), and an isolated complementarity determining region (CDR). These antibody fragments can be obtained using conventional techniques known to those with skill in the art, and the fragments can be screened for utility in the same manner as are intact antibodies.

Antibodies or fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., 1995, J. Immunol. Methods 182:41-50; WO 92/22324; WO 98/46645). Chimeric antibodies can be produced using the methods described in, e.g., Morrison, 1985, Science 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods 248:1-6 (2001); and Tutt et al., J. Immunol. 147: 60 (1991).

Other Polypeptide Targeting Moieties

Other polypeptides may be used as targeting moieties, e.g., antibody-like scaffold proteins or antibody mimetics such as fibronectin type III domains, DARPins, or centyrins. In some embodiments, the targeting moiety is a non-antibody protein scaffold, such as a knottin, an affibody, a green fluorescent protein, an ankryn repreat protein, an SH2 domain, or a PDZ domain. Exemplary protein scaffolds of the invention are described in Stern, L. A., et al., Curr. Opion. in Chem. Engineering, 2013, 2:425-432; Banta, S. et al., Annu. Rev. Biomed. Eng., 2013, 15:93-113; Skrlec, K., Trends in Biotechnology, 2015, 33(7):408-418; and Huang, H. et al., Methods Mol. Biol. 2017, 1555:225-254, each of which is incorporated herein with respect to the protein scaffolds therein.

In some embodiments, polypeptide targeting moieties may include monobodies constructed using a fibronectin type III domain (e.g., tenth type III domain of human fibronectin). In some embodiments, the polypeptide targeting moiety is a tenth type III domain of human fibronectin, or a polypeptide variant thereof, having at least 80% sequence identity with SEQ ID NO: 1.

SEQ ID NO: 1
VSDVPRDLEVVAATPTSLLISWDAPAVTVRYYRITYGETGGNSPVQEFTV
PGSKSTATISGLKPGVDYTITVYAVTGRGDSPASSKPISINYRT

In some embodiments, the polypeptide targeting moiety is an engineered or diversified polypeptide variant of SEQ ID NO: 1. For example, the polypeptide targeting moiety may be an engineered or diversified variant of the fibronectin type III domain of SEQ ID NO: 1, wherein the polypeptide variant includes mutations to one or more amino acids of the peptide (e.g., one or more solvent exposed amino acids of the polypeptide). In particular, loops BC, DE, and FG of the tenth type III domain of human fibronectin (bold in SEQ ID NO. 1) are structurally analogous to the antibody complementarity-determining regions H1, H2, and H3, respectively, and may be diversified or engineered to select for binding to a target (e.g., a polypeptide target). Three additional loops, AB, CD, and EF, are also candidates for engineering or diversification. See, for example, Lipovsek, D. Protein Eng Des Sel. 24(1-2):3-9 (2011).

In some embodiments, the polypeptide targeting moiety in an engineered or diversified variant of the tenth type III domain of human fibronectin, where the BC, DE, or FG loop regions may include one or more mutations relative to SEQ ID NO: 1. In some embodiments, the polypeptide targeting moiety is an engineered or diversified variant of the tenth type III domain of human fibronectin, having at least 80% sequence identity SEQ ID NO: 2, where “X” is any amino acid, and “m,” “n,” and “k” each, independently, have a value of 2 or greater (e.g., 3, 4, 5, 6, 7, 8, 9, 10, 11, 12, 13, 14, 15, 16, 17, 18, 19, 20).

SEQ ID NO: 2
VSDVPRDLEVVAATPTSLLISWD(X)ₘYYRITYGETGGNSPVQEFTVPG
(X)ₙATISGLKPGVDYTITVYAV(X)ₖSSKPISINYRT

Libraries of antibody-like scaffold proteins (e.g., fibronectin type III domains) may be generated using molecular display and directed evolution techniques known in the art such as phage display, DNA or RNA display, or yeast surface display.

Protein Variants

A protein or polypeptide variant, as described herein, generally has an amino acid sequence that shows significant (e.g., 80% or more, i.e., 80%, 81%, 82%, 83%, 84%, 85%, 86%, 87%, 88%, 89%, 90%, 91%, 92%, 93%, 94%, 95%, 96%, 97%, 98%, 99%, or more) identity with that of a reference polypeptide but includes a limited number of particular amino acid changes (e.g., insertions, deletions, or substitutions, either conservative or non-conservative and/or including one or more amino acid variants or analogs [e.g., D-amino acids, desamino acids]) relative to the reference polypeptide. In certain embodiments, a variant shares a relevant biological activity (e.g., binding to a particular compound or moiety thereof) with the reference polypeptide; in some such embodiments, the variant displays such activity at a level that is not less than about 50% of that of the reference polypeptide and/or is not less than about 0.5 fold below that of the reference polypeptide.

In some embodiments, a variant polypeptide has an amino acid sequence that differs from that of a reference polypeptide at least (or only) in that the variant has a larger number of cysteine residues and/or has one or more cysteine residues at a position corresponding to a non-cysteine residue in the reference polypeptide. For example, in some embodiments, addition of one or more cysteine residues to the amino or carboxy terminus of any of a polypeptide as described herein can facilitate conjugation of such polypeptide by, e.g., disulfide bonding.

In some embodiments, amino acid substitutions can be conservative (i.e., wherein a residue is replaced by another of the same general type or group) or non-conservative (i.e., wherein a residue is replaced by an amino acid of another type). In some embodiments, a naturally occurring amino acid can be substituted for a non-naturally occurring amino acid (i.e., non-naturally occurring conservative amino acid substitution or a non-naturally occurring non-conservative amino acid substitution), or vice versa.

Polypeptides made synthetically can include substitutions of amino acids not naturally encoded by DNA (e.g., non-naturally occurring or unnatural amino acid). Examples of non-naturally occurring amino acids include D-amino acids, an amino acid having an azide-containing side chain, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the structure (e.g., a local structure or global structure) of the original protein. Examples of substitutions identified as “conservative substitutions” are shown in Table 1. If such substitutions result in a change not desired, then other type of substitutions, denominated “exemplary substitutions” in Table 1, or as further described herein in reference to amino acid classes, are introduced and the products screened.

Substantial modifications in function or immunological identity are accomplished by selecting substitutions that differ significantly in their effect on maintaining (a) the structure of the protein backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),
(2) neutral hydrophilic: Cysteine (Cys), Serine (Ser), Threonine (Thr)
(3) acidic/negatively charged: Aspartic acid (Asp), Glutamic acid (Glu)
(4) basic: Asparagine (Asn), Glutamine (Gin), Histidine (His), Lysine (Lys), Arginine (Arg)
(5) residues that influence chain orientation: Glycine (Gly), Proline (Pro);
(6) aromatic: Tryptophan (Trp), Tyrosine (Tyr), Phenylalanine (Phe), Histidine (His),
(7) polar: Ser, Thr, Asn, Gln
(8) basic positively charged: Arg, Lys, His, and;
(9) charged: Asp, Glu, Arg, Lys, His
   Other amino acid substitutions are listed in Table 1.

TABLE 1
Amino acid substitutions

| Original | Exemplary | Conservative |
|---|---|---|
| residue | substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

Protein Variants with Altered Reactive Amino Acid Profiles

In some embodiments, a protein or polypeptide variant may include the addition of one or more reactive amino acid residues (e.g., cysteines) to a protein (e.g., at the amino or carboxy terminus of any of the proteins described herein) can facilitate conjugation of these proteins by, e.g., disulfide bonding. In some embodiments, one or more reactive amino acids (e.g., cysteines) may be removed to decrease the number of possible conjugation sites on a protein. Amino acid substitutions can be conservative (i.e., wherein a residue is replaced by another of the same general type or group) or non-conservative (i.e., wherein a residue is replaced by an amino acid of another type). In addition, a naturally occurring amino acid can be substituted for a non-naturally occurring amino acid (i.e., non-naturally occurring conservative amino acid substitution or a non-naturally occurring non-conservative amino acid substitution).

### Libraries

In some embodiments, the compounds, small molecule moieties, and/or polypeptide targeting moieties of the invention are members of libraries. Libraries including the compounds, small molecule moieties, and/or polypeptide targeting moieties of the invention may be tagged to assist in identification of library members. For example, the libraries may be tagged using any method known in the art such as DNA display libraries (also known as DNA-encoded libraries), RNA display libraries, yeast display libraries, or phage display libraries. Tagged libraries offer advantages such as allowing the screening of large libraries in one pot. In some embodiments, the compounds, small molecule moieties, and/or polypeptide targeting moieties of the invention are members of large libraries (e.g., libraries including at least 10⁵ members). In some embodiments, a library of the invention includes between about 10² to 10²⁰ complexes (e.g., about 10² to 10³, 10² to 10⁴, 10² to 10⁵, 10² to 10⁶, 10² to 10⁷, 10² to 10⁸, 10² to 10⁶, 10² to 10¹⁰, 10² to 10¹¹, 10² to 10¹², 10² to 10¹³, 10² to 10¹⁴, 10² to 10¹⁵, 10² to 10¹⁶, 10² to 10¹⁷, 10² to 10¹⁸, 10² to 10¹⁶, 10⁴ to 10⁵, 10⁴ to 10⁶, 10⁴ to 10⁷, 10⁴ to 10⁸, 10⁴ to 10⁶, 10⁴ to 10¹⁰, 10⁴ to 10¹¹, 10⁴ to 10¹², 10⁴ to 10¹³, 10⁴ to 10¹⁴, 10⁴ to 10¹⁵, 10⁴ to 10¹⁶, 10⁴ to 10¹⁷, 10⁴ to 10¹⁸, 10⁴ to 10¹⁶, 10⁴ to 10²⁰, 10⁵ to 10⁶, 10⁵ to 10⁷, 10⁵ to 10⁸, 10⁵ to 10⁶, 10⁵ to 10¹⁰, 10⁵ to 10¹¹, 10⁵ to 10¹², 10⁵ to 10¹³, 10⁵ to 10¹⁴, 10⁵ to 10¹⁵, 10⁵ to 10¹⁶, 10⁵ to 10¹⁷, 10⁵ to 10¹⁸, 10⁵ to 10¹⁹, or 10⁵ to 10²⁰ members.

Library Generation

Libraries useful in the methods of the invention may be prepared using any method known in the art. In some embodiments, libraries of polypeptide targeting moieties (e.g., fibronectin type III domains) may be prepared by a designed sitewise diversification strategy utilizing high throughput evolution and bioinformatics. In some embodiments, the members of the library may be prepared with only one reactive amino acid residue or with one optimal reactive amino acid residue. For example, libraries of polypeptide targeting moieties may be prepared using methods similar to those described in Woldring et al. PLoS One 10:e0138956 (2015), the methods of which are herein incorporated by reference. In some embodiments, the library of polypeptide targeting moieties is a synthetic antibody library. Synthetic antibody libraries may be prepared using any method known in the art. For example, a synthetic antibody library may be prepared by utilizing a CDR randomization method such as those described in Chen et al. Methods in Mol. Biol. 1131:113-131 (2014) and Mandrup et al. PLoS One 8(10):e76834 (2013), the methods of which are herein incorporated by reference. The libraries of polypeptide targeting moieties may be DNA or RNA display libraries, yeast display libraries, or phage display libraries.

Libraries of polypeptide targeting moieties may be conjugated to one or more small molecule moieties to prepare a library of bifunctional compounds for use in the methods of the invention. The polypeptide targeting moieties may be conjugated to the one or more small molecule moieties via a linker (e.g., a polyethylene-containing linker). In some embodiments, all of the polypeptide targeting moieties in the library are conjugated to the same small molecule moiety. In some embodiments, all of the polypeptide targeting moieties in the library are conjugated to the small molecule moiety with the same linker. In some embodiments, the polypeptide targeting moieties in the library are conjugated to a small molecule moiety with different linkers.

Library Screening

The libraries of bifunctional compounds of the invention may be screened for the ability to modulate the activity of a target protein using any method known in the art. In some embodiments, libraries of bifunctional compound may be screened for activity using flow cytometry methods, magnetic selection methods, or pull down methods. In some embodiments, the libraries of bifunctional compounds may be screened for activity using fluorescence based competition assays. For example, libraries of bifunctional compounds may be screened using methods described in Cho et al. Protein Eng. Des. Sel. 23:567-577 (2010); Tillotson, et al. Methods 60:27-37 (2013); Wang et al. J. Immunol. Methods 304:30-42 (2005); Wang et al. Nat. Methods 4:143-145 (2007); or Hoogenboom Nature Biotech. 23:1105-1116 (2005), the methods of each of which are herein incorporated by reference. The identity of the bifunctional compounds in the library with optimal activity may be determined utilizing any method known in the art. For example, if the bifunctional compound library is a DNA or RNA display library, the identity of the bifunctional compounds may be determined by sequencing the DNA or RNA tag on each compound. If the bifunctional compound library is a yeast display library, the identity of the bifunctional compound may be determined by sequencing the enriched yeast plasmid.

### Target Proteins

A target protein (e.g., a eukaryotic target protein such as a mammalian target protein or a fungal target protein or a prokaryotic target protein such as a bacterial target protein) is any protein whose activity is desirably altered. In one example, a target protein mediates a disease condition or a symptom of a disease condition. As such, a desirable therapeutic effect can be achieved by modulating (inhibiting or increasing) its activity.

Target proteins can be naturally occurring, e.g., wild type. Alternatively, a target protein can vary from the wild type protein but still retain biological function, e.g., as an allelic variant, a splice mutant or a biologically active fragment. In some embodiments, a target protein is a transmembrane protein.

In some embodiments, the target protein is a GPCR. In some embodiments, the target protein is a Rhodopsin-like receptor such as a protein encoded by the gene CCR1; CCR2; CCR3; CCR4; CCR5; CCR8; CCRL2; XCR1; CX3CR1; GPR137B; CCRL1; CCR6; CCR7; CCR9; CCR10; CXCR3; CXCR4; CXCR5; CXCR6; CXCR7; IL8RA; IL8RB; GPR182; DARC; GPER; AGTR1; AGTR2; AGTRL1; BDKRB1; BDKRB2; GPR15; GRP25; OPRD1; OPRK1; OPRM1; OPRL1; SSTR1; SSTR2; SSTR3; SSTR4; SSTR5; NPBWR1; NPBWR2; GPR1; GALR1; GALR2; GALR3; CYSLTR1; CYSLTR2; LTB4R; LTB4R2; RXFP1; RXFP2; RXFP3; RXFP4; KISS1R; MCHR1; UTS2R; CCKAR; CCKBR; NPFFR1; NPFFR2; HCRTR1; HCRTR2; AVPR1A; AVPR1B; AVPR2; GNRHR; QRFPR; GPR22; GPR176; BRS3; NMBR; GRPR; EDNRA; EDNRB; GPR37; NMUR1; NMUR2; NTSR1; NTSR2; TRHR; GHSR; GPR39; MLNR; C3AR1; C5AR1; CMKLR1; FPR1; FPRL1; FPRL2; MAS1; MAS1L; GPR1; GPR32; GPR44; GPR77; MTNR1A; MTNR1B; TACR1; TACR2; TACR3; NPYR; NPY2R; PPYR1; NPYSR; PRLHR; PROKR1; PROKR2; GPR19; GPR50; GPR75; GPR83; FSHR; LHCGR; TSHR; LGR4; LGR5; LGR6; FFAR1; FFAR2; FFAR3; GPR42; P2RY1; P2RY2; P2RY4; P2RY6; P2RY8; P2RY11; HCAR1; HCAR2; HCAR3; GPR31; GPR82; OXFR1; SUCNR1; P2RY12; P2RY13; P2RY14; GPR34; GPR87; GPR171; PTAFR; CNR1; CNR2; LPAR1; LPAR2; LPAR3; S1PR1; S1PR2; S1PR3; S1PR4; S1PR5; MC1R; MC3R; MC4R; MC5R; MC2R; GPR3; GPR6; GPR12; PTGDR; PTGER1; PTGER2; PTGER3; PTGER4; PTGFR; PTGIR; TBXA2R; LPAR4; LPAR5; LPAR6; P2RY10; F2RL1; F2RL2; F2RL3; GPR183; GPR4; GPR65; GPR68; GPR17; GPR18; GPR20; GPR35; GPR55; F2R; RHO; OPN1SW; OPN1MW; OPN1LW; OPN3; OPN4; OPN5; RGR; RRH; 5-HT; HT2RA; HT2RB; HT2RC; HTR6; ADRA1A; ADRA1B; ADRA1D; ADRA2A; DARA2B; ADRA2C; ADRB1; ADRB2; ADRB3; DRD1; DRD2; DRD3; DRD4; DRD5; TAAR1; TAAR2; TAAR3; TAAR5; TAAR6; TAAR8; TAAR9; HRH2; HRH1; HRH3; HRH4; ADORA1; ADORA2A; ADORA2B; ADORA3; CHRM1; CHRM2; CHRM3; CHRM4; CHRM5; GPR21; GPR27; GPR45; GPR52; GPR61; GPR63; GPR78; GPR84; GPR85; GPR88; GPR101; GPR161; GPR173; HTR1A; HTR1B; HTR1D; HTR1E; HTR1F; HTR4; HTR5A; HTR7; VN1R1; VN1R2; VN1R3; VN1R4; or VN1R5. In some embodiments, the target protein is a Secretin receptor such as a protein encoded by the gene ADCYAP1R1; CALCR; CRHR1; CRHR2; GIPR; GCGR; GLP1R; GLP2R; GHRHR; PTHR1; PTHR2; SCTR; VIPR1; VIPR2; BAI1; BAI2; BAI3; CD97; CELSR1; CELSR2; CELSR3; EMR1; EMR2; EMR3; EMR4; CPR56; CPR64; GPR97; GPR110; GPR111; GPR112; GPR113; GPR114; GPR115; GPR123; GPR125; GPR126; GPR128; GPR133; GPR144; GPR157; ELTD1; LPHN1; LPHN2; LPHN3; GPR116; HCTR-5; HCTR-6; KPG 006; or KPG 008. In some embodiments, the target protein is a metabotropic glutamate receptor such as a protein encoded by the gene GRM1; GRM5; GRM2; GRM3; GRM4; GRM6; GRM7; or GRM8. In some embodiments, the target protein is a cyclic AMP receptor. In some embodiments, the target protein is a Frizzled receptor family such as a protein encoded by the gene FZD1; FZD2; FZD3; FZD4; FZD5; FZD6; FZD7; FZD8; FZD9; or FZD10. In some embodiments, the target protein is smoothened.

In some embodiments, the target protein is an ion channel. In some embodiments, the target protein is a calcium activated potassium channel such as a protein encoded by the gene KCNMA1; KCNC1; KCNN2; KCNN3; KCNN4; KCNT1; KCNT2; or KCNU1. In some embodiments, the target protein is a CatSper and Two-pore channel such as a protein encoded by the gene CATPSER1; CATSPER2; CATSPER3; CATSPER4; TPCN1; or TPCN2. In some embodiments, the target protein is a cyclic nucleotide-regulated channels such as a protein encoded by the gene CNGA1; CNGA2; CNGA3; CNGA4; CNGB1; CNGB3; HCN1; HCN2; HCN3; or HCN4. In some embodiments, the target protein is an inwardly rectifying potassium channel such as a protein encoded by the gene KCNJ1; KCNJ2; KCNJ12; KCNJ4; KCNJ14; KCNJ3; KCNJ6; KCNJ9; KCNJ5; KCNJ10; KCNJ15; KCNJ16; KCNJ8; KCNJ11; or KCNJ13. In some embodiments, the target protein is a ryanodine receptor such as a protein encoded by the gene RYR1; RYR2; or RYR3. In some embodiments, the target protein is a transient receptor potential channel such as a protein encoded by the gene TRPAA1; TRPC1; TRPC2; TRPC3; TRPC4; TRPC5; TRPC6; TRPC7; TRPM1; TRPM2; TRPM3; TRPM4; TRPM5; TRPM6; TRPM7; TRPM8; MCOLN1; MCOLN2; MCOLN3; PKD2; PKD2L1; PDK2L2; TRPV1; TRPV2; TRPV3; TRPV4; TRPV5; or TRPV6. In some embodiments, the target protein is a two-P potassium channel such as a protein encoded by the gene KCNK1; KCNK2; KCNK3; KCNK4; KCNK5; KCNK6; KCNK7; KCNK9; KCNK10; KCNK12; KCNK13; KCNK15; KCNK16; KCNK17; or KCNK18. In some embodiments, the target protein is a voltage-gated calcium channel such as a protein encoded by the gene CACNA1S; CACNA1C; CACNA1D; CACNA1F; CACNA1A; CACNA1B; CACNA1E; CACNA1G; CACNA1H; or CACNA1I. In some embodiments, the target protein is a voltage-gated potassium channel such as a protein encoded by the gene KCNA1; KCNA2; KCNA3; KCNA4; KCNA5; KCNA6; KCNA7; KCNA10; KCNB1; KCNB2; KCNC1; KCNC2; KCNC3; KCNC4; KCND1; KCND2; KCND3; KCNF1; KCNG1; KCNG1; KCNG2; KCNG3; KCNG4; KCNQ1; KCNA2; KCNA3; KCNA4; KCNQ5; KCNV1; KCNV2; KCNS1; KCNS2; KCNS3; KCNH1; KCNH5; KCNH2; KCNH6; KCNH7; KCNH8; KCNH3; or KCNH4. In some embodiments, the target protein is a voltage-gated proton channel such as a protein encoded by the gene HVCN1. In some embodiments, the target protein is a voltage-gated sodium channel such as a protein encoded by the gene SCN1A; SCN2A; SCN3A; SCN4A; SCN5A; SCN8A; SCN9A; SCN10A; or SCN11A.

In some embodiments, the target protein is an enzyme. In some embodiments, the target protein is a oxidoreductase, dehydrogenase, luciferase, DMSO reductase, alcohol dehydrogenase (NAD), alcohol dehydrogenase (NADP), homoserine dehydrogenase, aminopropanol oxidoreductase, diacetyl reductase, glycerol dehydrogenase, propanediol-phosphate dehydrogenase, glycerol-3-phosphate dehydrogenase (NAD+), D-xylulose reductase, L-xylulose reductase, lactate dehydrogenase, malate dehydrogenase, isocitrate dehydrogenase, HMG-CoA reductase, glucose oxidase, L-gulonolactone oxidase, thiamine oxidase, xanthine oxidase, acetaldehyde dehydrogenase, glyceraldehyde 3-phosphate dehydrogenase, pyruvate dehydrogenase, oxoglutarate dehydrogenase, biliverdin reductase, protoporphyrinogen oxidase, monoamine oxidase, dihydrofolate reductase, methylenetetrahydrofolate reductase, sarcosine oxidase, dihydrobenzophenanthridine oxidase, NADH dehydrogenase, urate oxidase, nitrite reductase, nitrate reductase, glutathione reductase, thioredoxin reductase, sulfite oxidase, cytochrome c oxidase, coenzyme Q-cytochrome c reductase, catechol oxidase, laccase, cytochrome c peroxidase, catalase, myeloperoxidase, thyroid peroxidase, glutathione peroxidase, 4-hydroxyphenylpyruvate dioxygenase, renilla-luciferin 2-monooxygenase, cypridina-luciferin 2-monooxygenase, firefly luciferase, watasenia-luciferin 2-monooxygenase, oplophorus-luciferin 2-monooxygenase, cytochrome P450 oxidase, cytochrome P450, aromatase, a protein encoded by the gene CYP2D6, CYP2E1, or CYP3A4, cytochrome P450 oxidase, nitric oxide dioxygenase, nitric oxide synthase, aromatase, a protein encoded by the gen CYP2D6, CYP2E1, or CYP3A4, phenylalanine hydroxylase, tyrosinase, superoxide dismutase, ceruloplasmin, nitrogenase, deiodinase, glutathione S-transferase, Catechol-O-methyl transferase, DNA methyltransferase, histone methyltransferase, ornithine transcarbamoylase, aminolevulinic acid synthase, choline acetyltransferase, Factor XIII, gamma glutamyl transpeptidase, transglutaminase, hypoxanthine-guanine phosphoribosyltransferase, thiaminase, alanine transaminase, aspartate transaminase, butyrate kinase, hydrolytic enzyme, nuclease, endonuclease, exonuclease, acid hydrolase, phospholipase A, acetylcholinesterase, cholinesterase, lipoprotein lipase, ubiquitin carboxy-terminal hydrolase L1, phosphatase, alkaline phosphatase, fructose bisphosphatase, phospholipase C, cGMP specific phosphodiesterase type 5, phospholipase D, restriction enzyme Type 1, deoxyribonuclease I, RNase H, ribonuclease, amylase, sucrase, chitinase, lysozyme, maltase, lactase, beta-galactosidase, hyaluronidase, adenosylmethionine hydrolase, S-adenosyl-L-homocysteine hydrolase, alkenylglycerophosphocholine hydrolase, alkenylglycerophosphoethanolamine hydrolase, cholesterol-5,6-oxide hydrolase, hepoxilin-epoxide hydrolase, isochorismatase, leukotriene-A4 hydrolase, limonene-1,2-epoxide hydrolase, microsomal epoxide hydrolase, trans-epoxysuccinate hydrolase, alanine aminopeptidase, angiotensin converting enzyme, serine protease, chymotrypsin, trypsin, thrombin, Factor X, plasmin, acrosin, Factor VII, Factor IX, prolyl oligopeptidase, Factor XI, elastase, Factor XII, proteinase K, tissue plasminogen activator, Protein C, separase, pepsin, rennet, renin, trypsinogen, plasmepsin, matrix metalloproteinase, metalloendopeptidase, urease, beta-lactamase, arginase, adenosine deaminase, GTP cyclohydrolase I, nitrilase, helicase, DnaB helicase, RecQ helicase, ATPase, NaKATPase, ATP synthase, kynureninase, ornithine decarboxylase, uridine monophosphate synthetase, aromatic-L-amino-acid decarboxylase, rubisCO, carbonic anhydrase, tryptophan synthase, phenylalanine ammonia-lyase, cystathionine gamma-lyase, cystathionine beta-lyase, leukotriene C4 synthase, dichloromethane dehalogenase, halohydrin dehalogenase, adenylate cyclase, guanylate cyclase, phenylalanine racemase (ATP-hydrolysing), serine racemase, mandelate racemase, UDP-glucose 4-epimerase, methylmalonyl CoA epimerase, a protein encoded by the gene FKBP1A, FKBP1B, FKBP2, FKBP3, FKBP5, FKBP6, FKBP8, FKBP9, FKBP10, FKBP52, or FKBPL, cyclophilin, parvulin, prolyl isomerase, 2-chloro-4-carboxymethylenebut-2-en-1,4-olide isomerase, beta-carotene isomerase, farnesol 2-isomerase, furylfuramide isomerase, linoleate isomerase, maleate isomerase, maleylacetoacetate isomerase, maleylpyruvate isomerase, parvulin, photoisomerase, prolycopene isomerase, prolyl isomerase, retinal isomerase, retinol isomerase, zeta-carotene isomerase, enoyl CoA isomerase, protein disulfide isomerase, phosphoglucomutase, muconate cycloisomerase, 3-carboxy-cis,cis-muconate cycloisomerase, tetrahydroxpteridine cycloisomerase, inositol-3-phosphate synthase, carboxy-cis,cis-muconate cyclase, chalcone isomerase, chloromuconate cycloisomerase, (+)-bornyl diphosphate synthase, cycloeucalenol cycloisomerase, alpha-pinene-oxide decyclase, dichloromuconate cycloisomerase, copalyl diphosphate synthase, ent-copalyl diphosphate synthase, syn-copalyl-diphosphate synthase, terpentedienyl-diphosphate synthase, halimadienyl-diphosphate synthase, (S)-beta-macrocarpene synthase, lycopene epsilon-cyclase, lycopene beta-cyclase, prosolanapyrone-III cycloisomerase, D-ribose pyranase, topoisomerase, 6-carboxytetrahydropterin synthase, FARSB, glutamine synthetase, argininosuccinate synthetase, CTP synthase, pyruvate carboxylase, acetyl-CoA carboxylase, or DNA ligase.

### Methods of Preparation

The compounds of the invention may be prepared using methods known in the art. For example, a small molecule moiety may be conjugated to a linker which includes a cross-linking group (e.g., a maleimide) to produce a compound of Formula VII:

A-L-B Formula VII

wherein A includes a small molecule moiety;
L is a linker; and
B is a cross-linking moiety.

The compound of Formula VII may then be reacted with a polypeptide targeting moiety including one or more reactive amino acid residues (e.g., a free cysteine, a lysine, or a non-natural amino acid). In some embodiments, the small molecule moiety is conjugated to the polypeptide targeting moiety in a site-specific manner. In some embodiments, the site of conjugation is a solvent exposed amino acid of the polypeptide targeting moiety. In some embodiments, the site of conjugation is a solvent exposed amino acid located within the polypeptide chain (e.g., a solvent exposed amino acid residue located in a solvent exposed loop). In some embodiments, the site of conjugation is located at or near the terminus (e.g., within 10 amino acid residues of the C-terminus or within 10 amino acid residues of the N-terminus) of the polypeptide. In some embodiments, the polypeptide targeting moiety has been modified to include a reactive amino acid residue at a specific site. In some embodiments, the polypeptide targeting moiety has been modified to include no more than one reactive amino acid residue or no more than one reactive residue of a particular type (e.g., no more than one cysteine, no more than one lysine). In some embodiments, when the polypeptide targeting moiety is an antibody, the small molecule moiety is conjugated via a reactive amino acid residue in a CDR (e.g., CDR1) of the antibody. In some embodiments, the small molecule moiety is conjugated to the polypeptide targeting moiety via a glycosylation site. In some embodiments, the small molecule moiety is conjugated to the polypeptide targeting moiety via a framework residue distinct from the antigen binding site.

In some embodiments, a free cysteine utilized to conjugate a small molecule moiety to a polypeptide targeting moiety is produced by reducing the polypeptide targeting moiety under conditions sufficient to reduce at least one disulfide bond (e.g., a disulfide bond in the CDR or a loop region of the polypeptide targeting moiety). For example, the small molecule moiety may be conjugated to the polypeptide targeting moiety using methods similar to those described in Badescu, et al. Bioconjug. Chem. 25(3):460-469 (2014); Badescu et al. Bioconjug. Chem. 25(6):1124-1136 (2014); Bryant et al. Mol. Pharm. 12(6):1872-1879 (2015); Schumacher et al. Org. Biomol. Chem. 7261-7269 (2014); or Bryden et al. Bioconjug. Chem. 611-617 (2014), the methods of each of which are herein incorporated by reference.

In some embodiments, a small molecule is conjugated to a polypeptide (e.g., via a linker) by enzymatic ligation. Enzymatic ligation may be performed by various techniques known to one in the art. For example, enzymatic ligation may be performed by (a) prenylation of CaaX motifs with protein farnesyltransferase (e.g., a described in Rose, M. W., et al., Biopolymers, 2005, 80, 164-171; and Hurwitz, H. I.; Casey, P. J. Curr. Topics Membr. 2002, 52, 531-550); (b) conjugation of [a]-LPETG and GGG-[b] with sortase A (e.g., as described in Levary, D., et al., PLOS One, 2011, 6, e18342); (c) ligation with lipoic acid ligase (Lpl) at Lpl acceptor peptide (e.g., as described in Fernández-Suárez M, et al., Nat Biotech. 2007, 25, 1483-1487); or (d) using a transglutaminase reaction (e.g., as described in Strop, P. et al., Chemistry and Biology, 2013, 20, 161-167).

### Uses

Treatment of Diseases or Disorders

Compounds described herein may be useful in the methods of treating diseases or disorders related to the target proteins described herein, and, while not bound by theory, are believed to exert their desirable effects through their ability to modulate (e.g., positively or negatively modulate) the activity of a target protein (e.g., a eukaryotic target protein such as a mammalian target protein or a fungal target protein or a prokaryotic target protein such as a bacterial target protein).

Pharmaceutical Compositions

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compounds described herein may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, compounds described herein may be used alone, or in combination with one or more other active agents. An example of other pharmaceuticals to combine with the compounds described herein would include pharmaceuticals for the treatment of the same indication. Another example of a potential pharmaceutical to combine with compounds described herein would include pharmaceuticals for the treatment of different yet associated or related symptoms or indications. Depending on the mode of administration, compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

Compounds of the invention may be prepared and used as pharmaceutical compositions comprising an effective amount of a compound described herein and a pharmaceutically acceptable carrier or excipient, as is well known in the art. In some embodiments, a composition includes at least two different pharmaceutically acceptable excipients or carriers.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluents as well as, in some cases, adjuvants, buffers, preservatives and the like. Compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

Each compound of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with nontoxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluents (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

Administration of each drug in a combination therapy, as described herein, can, independently, be one to four times daily for one day to one year, and may even be for the life of the subject. Chronic, long-term administration may be indicated.

### EXAMPLES

### Example 1. Determination of Optimal Binding Sites, Linkers, and Small Molecule Moieties

A non-binding fibronectin type III domain is mutated to contain a single cysteine at specific positions (e.g., G79, T28, R30, R78, D80, S53, T71, K63, or L19). The fibronectin type III domains are displayed on the surface of a yeast cell. For example, using the methods described in Chen et al. Methods Enzymol. 523:303-326 (2013) and/or Boder et al. Nat. Biotechnol. 15:553-557 (1997).

Small molecule moieties (e.g., sulfonamide-containing small molecule moieties) are conjugated to the mutated fibronectin type III domains via a cross-linking group (e.g., a maleimide). If more than one small molecule moiety is used, the bifunctional compounds with different small molecule moieties or different linkers may be separated.

The bifunctional compounds are combined with a target protein (e.g., a carbonic anhydrase such as carbonic anhydrase 9 or carbonic anhydrase 2) and the binding of the bifunctional compound to the target is determined, e.g., using a fluorescence based competition assay.

### Example 2. Preparation of Bifunctional Compound Libraries

Bifunctional compounds identified in Example 1 which are determined to bind to the target protein are utilized as starting points for libraries.

A library of fibronectin type III domains identified in Example 1 as resulting in bifunctional compounds capable of binding the target protein is prepared by a designed sitewise diversification strategy of the remainder of the paratope (e.g., diversification of any amino acid except the added cysteine) developed through high throughput evolution and bioinformatics. For example, diversification is conducted using methods described in Woldring et al. PLoS One 10:e0138956 (2015).

The library of fibronectin type III domains is introduced into the yeast display system by homologous recombination. The displayed fibronectin type III domains are conjugated to the small molecule moieties (e.g., via a cross-linking moiety such as a maleimide-containing cross-linking moiety).

### Example 3. Screening of Bifunctional Compound Against Target Protein

Bifunctional compounds are screened for activity against target proteins, e.g., using flow cytometry, magnetic selection, or yeast pull down assays. For example, bifunctional compounds are screened with flow cytometry with fluorescently labeled human lysate or yeast pull-down on adherent human cell monolayers as described in Cho et al. Protein Eng. Des. Sel. 23:567-577 (2010); Tillotson, et al. Methods 60:27-37 (2013); Wang et al. J. Immunol. Methods 304:30-42 (2005); or Wang et al. Nat. Methods 4:143-145 (2007).

The identity of bifunctional compounds which bind to the target protein are determined by sequencing enriched yeast plasmid.

### Example 4. Identification of Bifunctional Compounds which Bind Carbonic Anhydrase

Bifunctional compounds comprising a fibronectin type III domain (Fn) conjugated via a linker to acetazolamide (AAZ) were identified by screening yeast-displayed Fn-AAZ libraries for their ability to bind either carbonic anhydrase 9 (CA9) or carbonic anhydrase 2 (CA2). Selection of an Fn-AAZ library against CA9 produced Fn-AAZ clones that bind with high affinity to CA9, including Fn-AAZ clones that bind to CA9 with higher affinity than either Fn or AAZ, alone. Selected Fn-AAZ clones also showed increased specificity for CA9 over related carbonic anhydrase 2 (CA2). Selection of an Fn-AAZ library against CA2 produced Fn-AAZ clones that bind with high affinity to CA2.

Target Biotinylation

5 μM recombinant His-tagged human carbonic anhydrase 9 (CA9, Sino Biological) was incubated with 500 μM EZ-link NHS-Biotin (Thermo) in PBS, pH 7.4 for 4 hours at room temperature. Approximately 2 biotin molecules conjugated per CA9 molecule was confirmed by MALDI-TOF mass spectrometry. Carbonic anhydrase 2 (CA2, Sino Biological) was biotinylated in the same manner at 15 μM with 135 μM NHS-Biotin, and conjugation was again confirmed by MALDI-TOF. Unreacted biotin was removed by two sequential desaltings using Zeba 7K desalting spin columns (Thermo).

Yeast Display of Fibronectin Clones

Plasmids encoding single clones pCT-FnR (a hydrophilic fibronectin domain binder to rabbit IgG) and pCT-FnR-T28C were transformed into EBY100 yeast following the EZ yeast method (Zymo Research). Plasmids were generated using standard cloning techniques. Transformed yeast were grown by shaking at 30° C. in SD-CAA (16.8 g/L sodium citrate dihydrate, 3.9 g/L citric acid, 20.0 g/L dextrose, 6.7 g/L yeast nitrogen base, 5.0 g/L casamino acids) media and induced by transferring to SG-CAA (10.2 g/L sodium phosphate dibasic heptahydrate, 8.6 g/L sodium phosphate monobasic monohydrate, 19.0 g/L galactose, 1.0 g/L dextrose, 6.7 g/L yeast nitrogen base, 5.0 g/L casamino acids) media and shaking for at least 4 hours at 30° C.

Selection of Sites for Conjugation to Small Molecules

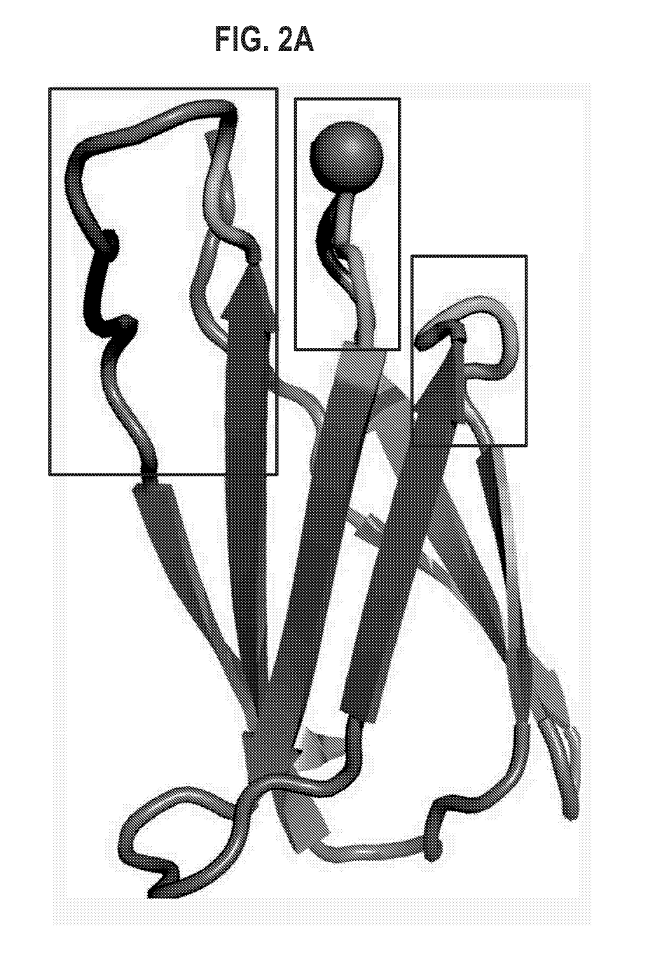

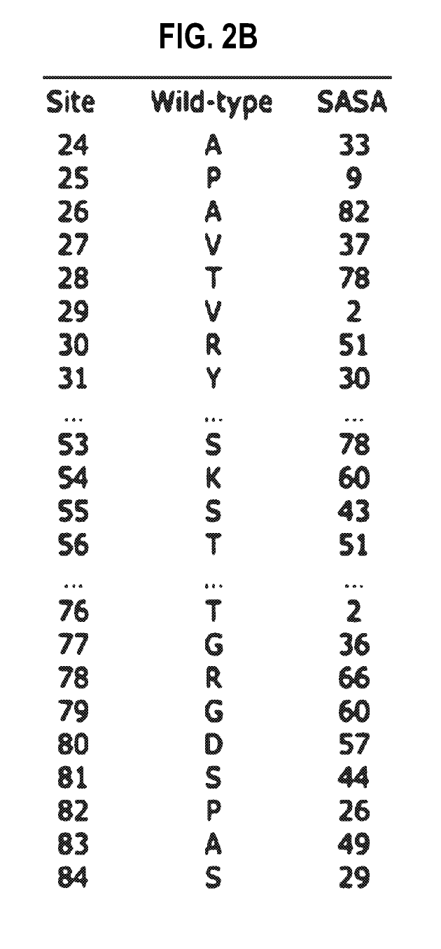

The structure of Fn was evaluated to select amino acids (e.g., solvent exposed amino acids) for conjugation to a small molecule (AAZ) (FIGS. 2A-B).

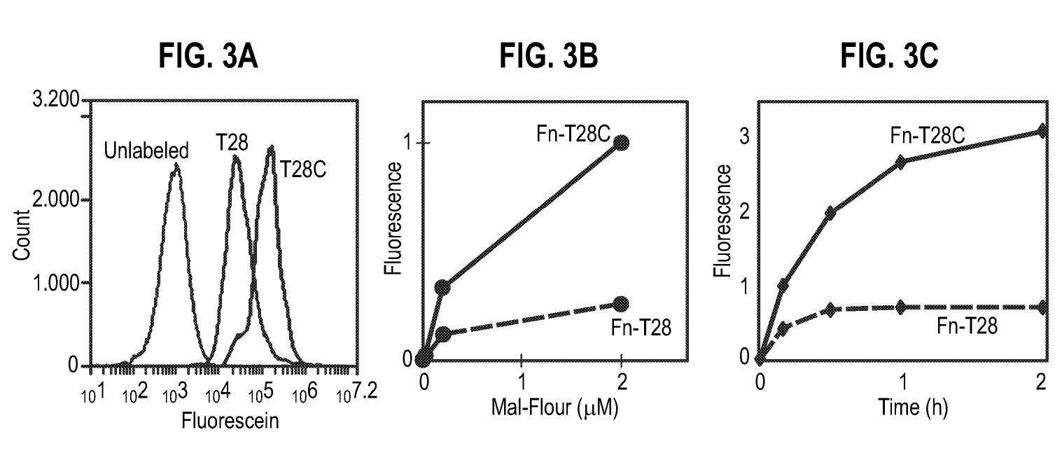

As shown in FIGS. 3A-C, Maleimide-fluorescein is effectively conjugated to yeast-displayed Fn with a single cysteine. EBY100 yeast, transformed with pCT-FnR or pCT-FnR-T28C vector, were induced to display the indicated Fn clone. Two million induced yeast were washed in PBS, pH 7.4 and incubated with maleimide-fluorescein in 50 μL PBS, pH 7.4 at room temperature. Yeast were washed with PBS +1 g/L BSA (PBSA) and analyzed via flow cytometry.

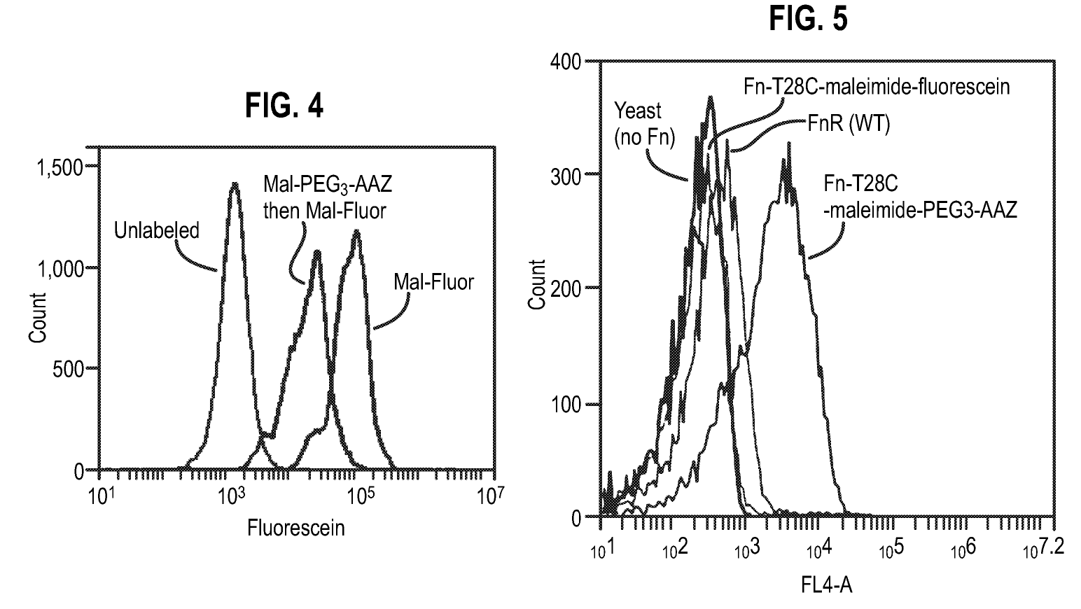

As shown in FIG. 4, Maleimide-AAZ is effectively conjugated to yeast-displayed Fn. EBY100 yeast, transformed with pCT-FnR-T28C vector, were induced to display the Fn clone. Two million induced yeast were washed in PBS, pH 7.4 and incubated with 0 or 2 μM maleimide-PEG3-AAZ in PBS, pH 7.4 for 2 hours. Yeast were then incubated with 2 μM maleimide-fluorescein in PBS, pH 7.4 for 2 hours. Yeast were washed with PBSA and analyzed via flow cytometry. Unlabeled yeast were included as a control. Conjugation with maleimide-AAZ reduced fluorescein conjugation by 70%, which is consistent with effective AAZ conjugation.

Single Clone and Library Conjugation to Small Molecules

Two million induced yeast expressing selected fibronectin clones were washed in PBS, pH 6.5, then incubated in 50 μL PBS, pH 6.5 with 0.02, 0.2, or 2 μM fluorescein-5-maleimide (Pierce) or maleimide-XPEG-Acetazolamide (AAZ) for 10 minutes, 30 minutes, 1, or 2 hours at room temperature while rocking. Yeast were washed 2× in 1 mL PBS, pH 7.4+1 g/L BSA (PBSA) to remove unconjugated small molecule. Library conjugations were performed on 3.4×10⁹ (15× diversity) transformed and induced yeast expressing the designed libraries in 2 mL PBS, pH 6.5 with 8 μM maleimide-XPEG-AAZ for 2 hours at room temperature while rocking. Small molecules with PEG linkers of lengths 2, 3, 5, and 7 were conjugated in separate reactions. After incubation, yeast were washed 4 times in 50 mL PBSA to remove unconjugated small molecule and the 4 conjugated yeast populations were pooled.

Library Construction

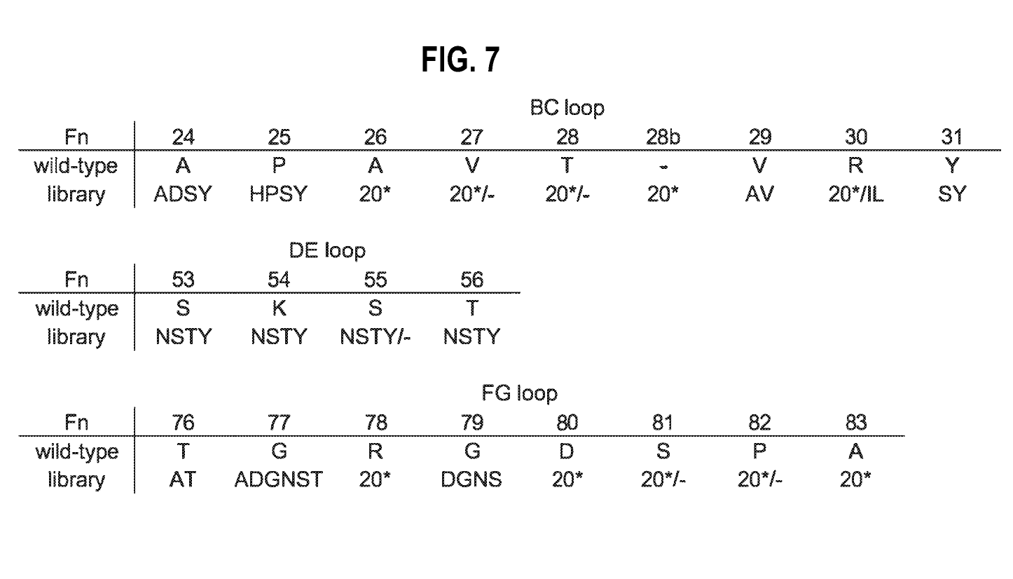

Oligonucleotides encoding the Fn-Cys yeast-displayed designed libraries were synthesized by IDT DNA Technologies, amplified by overlap extension PCR, and transformed into EBY100 by homologous recombination with the pCT vector following the protocol by Woldring, D. R., et al. PLoS One. 10:e0138956 (2015). As shown in FIG. 7, the indicated sites were diversified using degenerate codons to allow the indicated amino acids. 20* refers to a biased composition that balances amino acid frequencies observed in human antibody complementarity-determining regions as well as evolved fibronectin domains (Woldring, et al. PLOS One 2015). Loop lengths are varied by including or excluding codons at sites 27, 28, 55, 81, and 82. In one library, site 28 was conserved as cysteine. In a second library, site 80 was conserved as cysteine. Both libraries had 200 million transformants in yeast.

Sequence analysis confirms that library construction matches design.

Magnetic Bead Selection

15× diversity, small molecule conjugated libraries were incubated with 10 μL streptavidin coated dynabeads (Thermo) for 1 hour at room temperature while tumbling. Unbound yeast were removed and again incubated with 10 μL streptavidin coated Dynabeads. Unbound yeast were removed and incubated with 35.5 nM biotinylated CA9 in PBSA for 30 minutes at room temperature. Yeast were washed 4× in 2 mL PBSA, then incubated with 10 μL streptavidin coated Dynabeads in 1 mL PBSA for 1 hour at room temperature. Beads were washed 2× in PBSA, resuspended in SD-CAA media and incubated overnight while shaking at 30° C. Beads were then removed and the yeast were induced in SG-CAA for subsequent magnetic bead sorting, sorting by flow cytometry, or analysis.

Target Binding and Flow Cytometry

Fluorescein-5-maleimide conjugated to yeast-displayed FN was detected directly on a BD Accuri C6 flow cytometer using the standard equipped 488 nm excitation laser and 533/30 nm emission filter. In binding experiments, yeast-displaying Fn were conjugated with maleimide-XPEG-AAZ as described above. Yeast were incubated to equilibrium with indicated concentrations of biotinylated target at room temperature in sufficient volume of PBSA to ensure at least 10-fold molar excess of target to Fn. Primary antibody against c-MYC (9E10) was also included in the target incubation. Yeast were then washed in cold PBSA and incubated with streptavidin-AF647 (Thermo) and goat anti-mouse-FITC (Thermo) for 15 minutes at 4° C. Yeast were again washed in cold PBSA and analyzed using a BD Accuri C6 flow cytometer equipped with the standard 488 nm laser and 533/30 nm emission filter for FITC detection and the 640 nm laser and 675/25 nm emission filter for AF647 detection.

Yeast-Displayed Fn-AAZ is Functional at Multiple Conjugation Sites

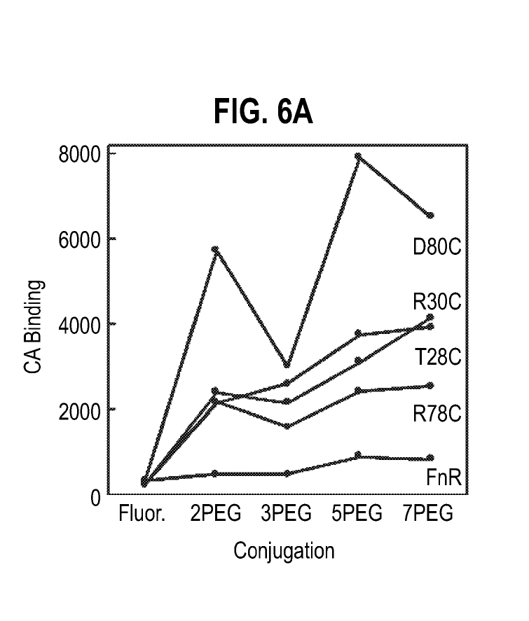

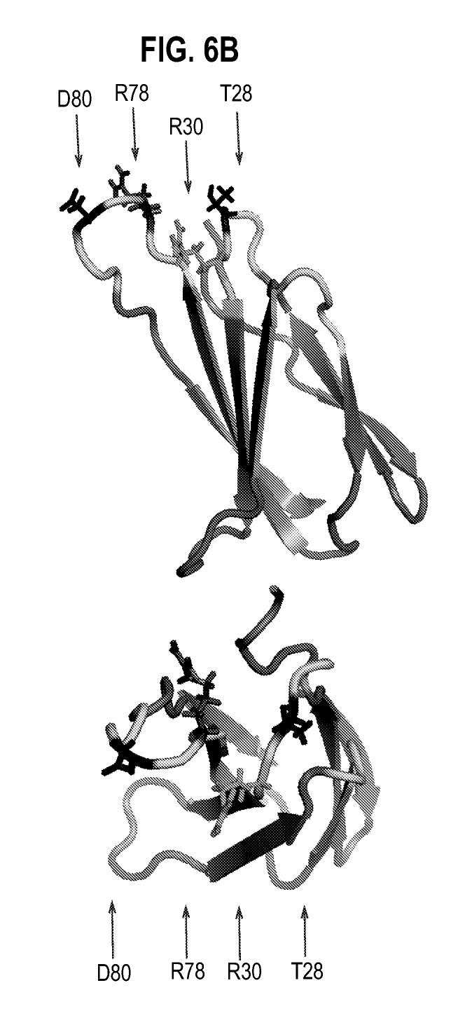

As shown in FIG. 6A-B, yeast-displayed Fn-AAZ is functional at multiple conjugation sites. Yeast displaying FnR or a single mutant (D80, R78, R30, or T28) were conjugated with maleimide-fluorescein or maleimide-PEG-AAZ. Yeast were washed with PBSA, incubated with 250 nM biotinylated carbonic anhydrase 9, washed with PBSA, and incubated with AlexaFluorconjugated streptavidin. Yeast were washed and analyzed by flow cytometry. All four conjugation sites exhibit functional binding.

Fn-AAZ Conjugates can be Selected for Binding to Carbonic Anhydrase 9

As shown in FIG. 5, yeast-displayed Fn-AAZ libraries were determined to bind carbonic anhydrase. Yeast displayed FnR or FnRT28C were conjugated with maleimide-fluorescein or maleimide-PEG3-AAZ. Yeast were washed with PBSA, incubated with 250 nM biotinylated carbonic anhydrase 9, washed with PBSA, and incubated with AlexaFluoroconjugated streptavidin. Yeast were washed and analyzed by flow cytometry. Bare yeast were also included for comparison. Dramatically increased carbonic anhydrase binding was observed for FnR-T28C+maleimide-PEG3-AAZ relative to both the non-cysteine control and the maleimide-fluorescein control.\

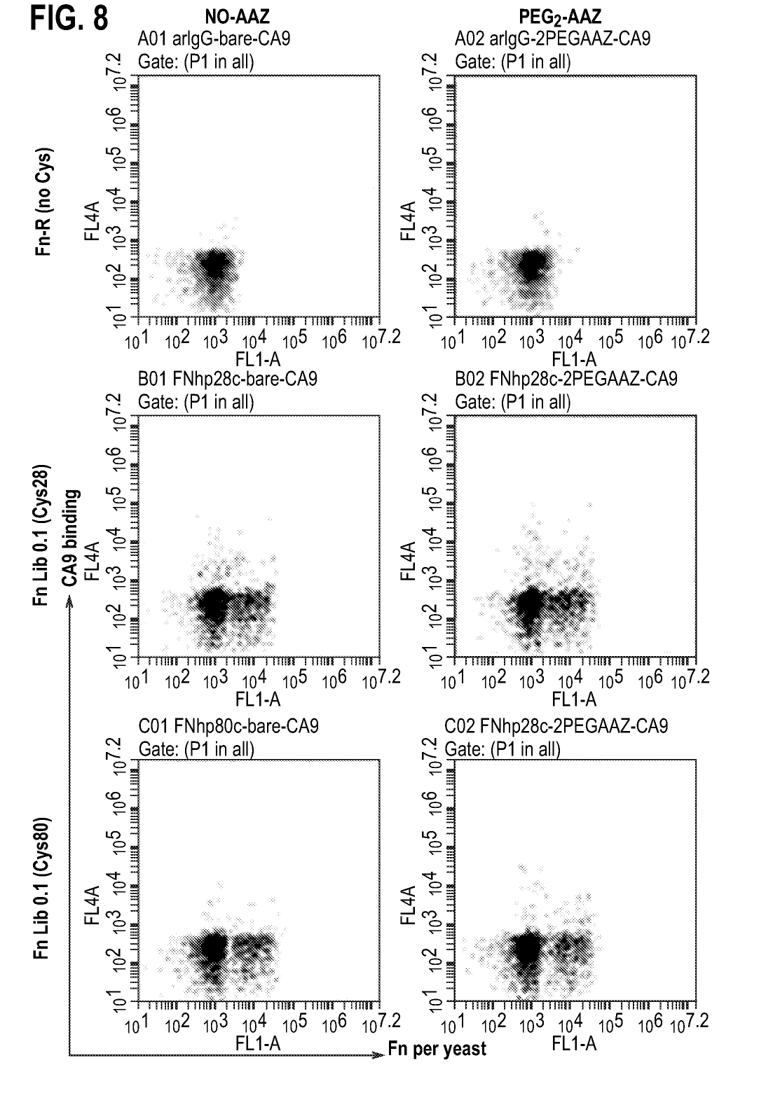

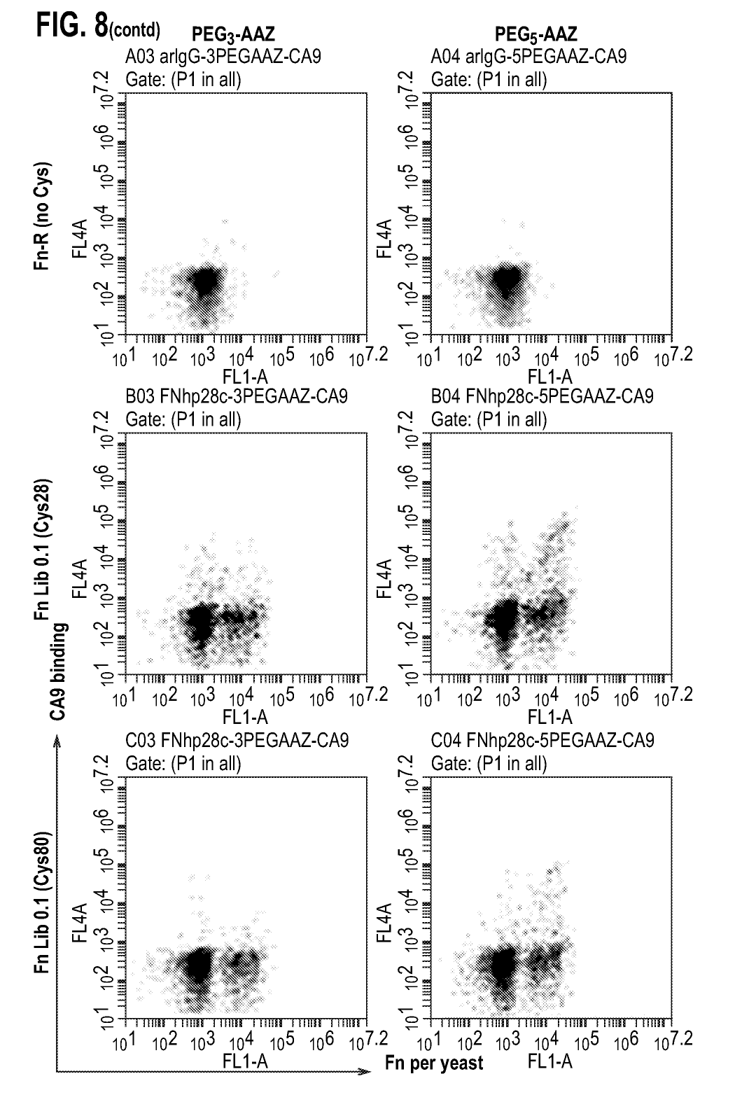

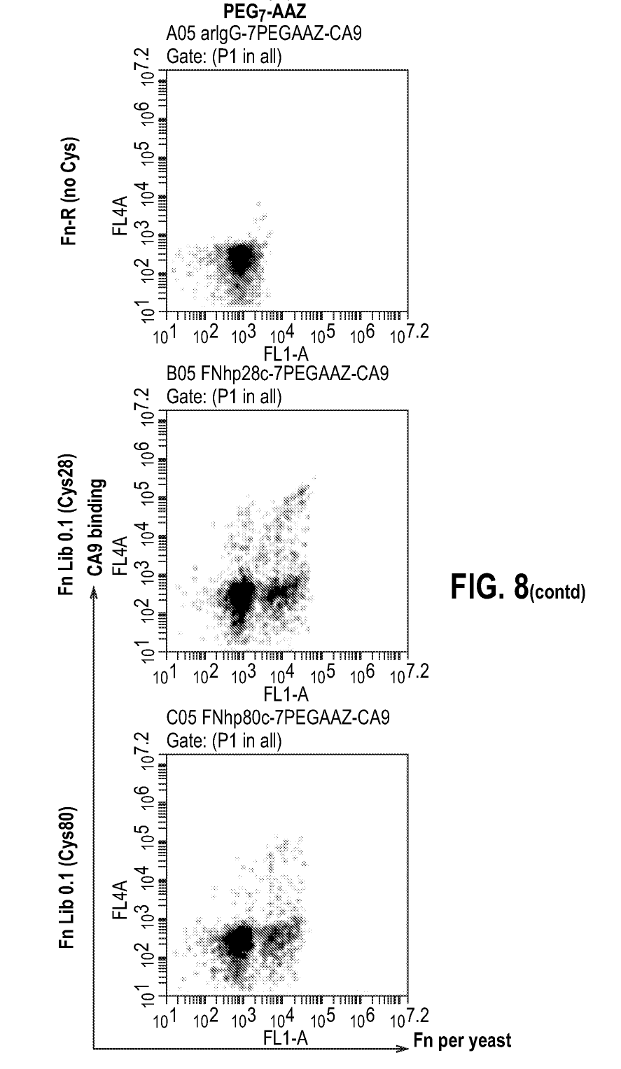

As shown in FIG. 8, Fn and AAZ provide mutual benefit in target binding. Yeast-displayed Fn libraries were sorted for binding to carbonic anhydrase using one magnetic bead selection (Ackerman, et al. Biotech. Prog. 2009). The resulting populations (Lib 0.1), as well as the FnR control, were conjugated with maleimide-PEG-AAZ (or not conjugated as a control). Yeast were labeled with 35.5 nM biotinylated carbonic anhydrase 9, washed, and labeled with AlexaFluor647-conjugated streptavidin. Yeast were also labeled with mouse anti-c-MYC antibody and AlexaFluor488-conjugated anti-mouse antibody to identify full-length Fn in the library populations (FnR lacks the c-MYC epitope). Binding is enabled by the combination of select Fn clones and the AAZ conjugation. AAZ conjugation by itself is insufficient to provide binding at this concentration in the context of FnR and many library variants. Fn variants by themselves (no AAZ) are unable to provide binding at this concentration.

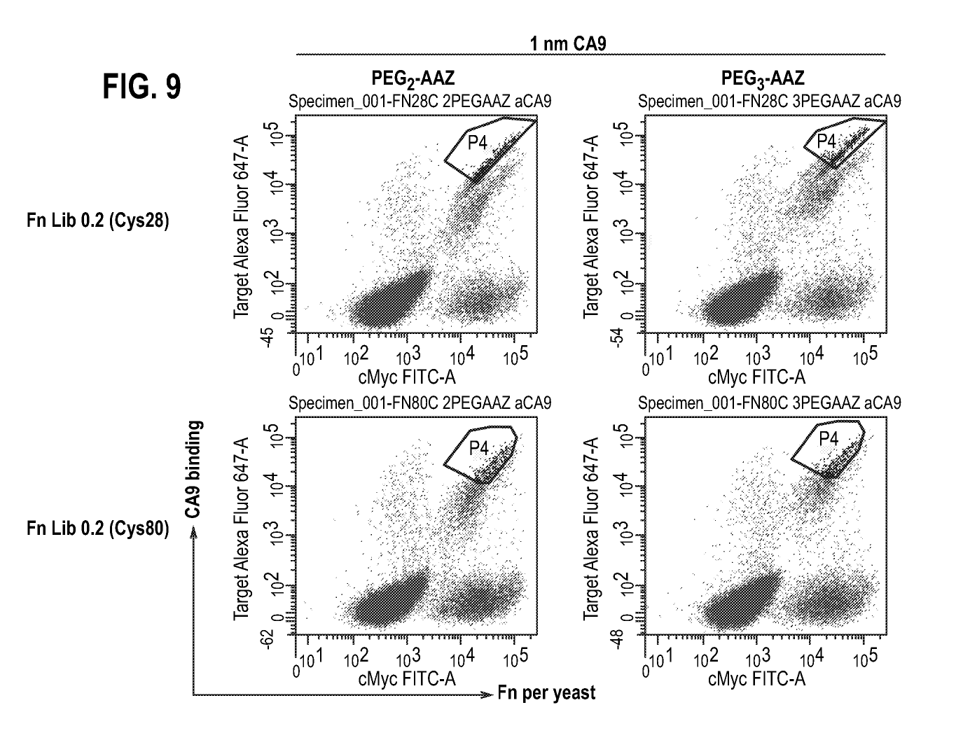

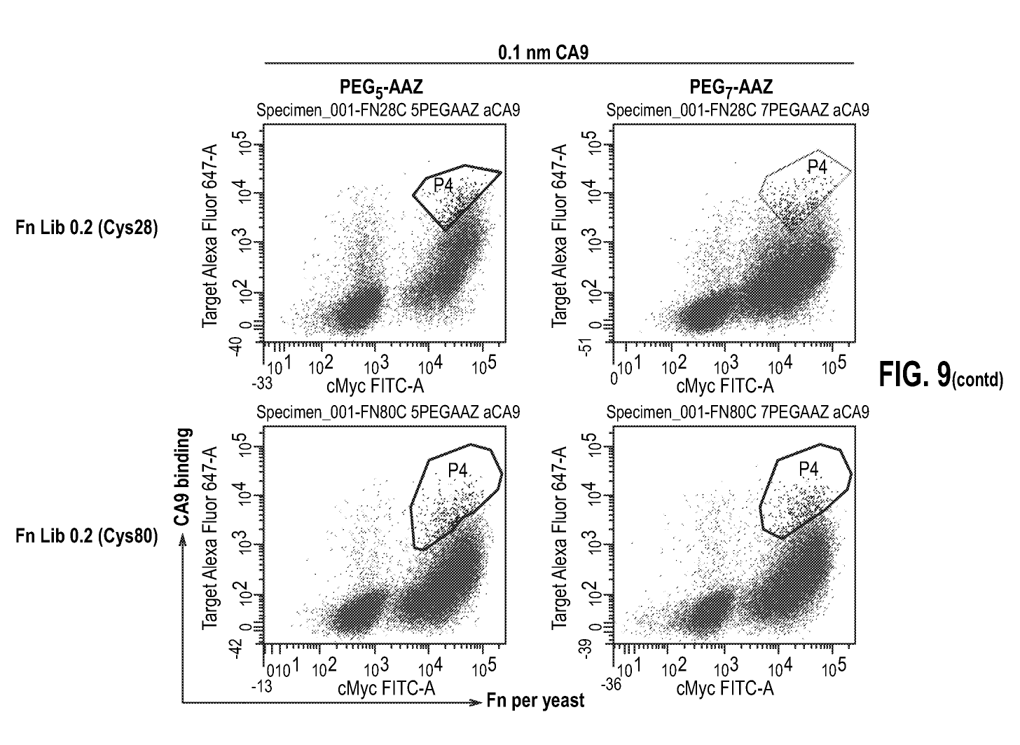

As shown in FIG. 9, Fn-AAZ conjugates bind strongly to carbonic anhydrase 9. Yeast-displayed Fn libraries were sorted twice for binding to carbonic anhydrase (two bead sorts for PEG2 and PEG3 populations; one bead sort and one flow cytometric sort for PEG5 and PEG7 populations). The resulting populations were conjugated with maleimide-PEG-AAZ. Yeast were labeled with 0.1 or 1 nM biotinylated carbonic anhydrase 9 (0.1 nM for PEG5 and PEG7; 1 nM for PEG2 and PEG3), washed, and labeled with AlexaFluor647-conjugated streptavidin. Yeast were also labeled with mouse anti-c-MYC antibody and AlexaFluor488-conjugated anti-mouse antibody to identify full-length Fn in the library populations.

Selection of Fn-AAZ Conjugates that Bind Selectively to Carbonic Anhydrase 9

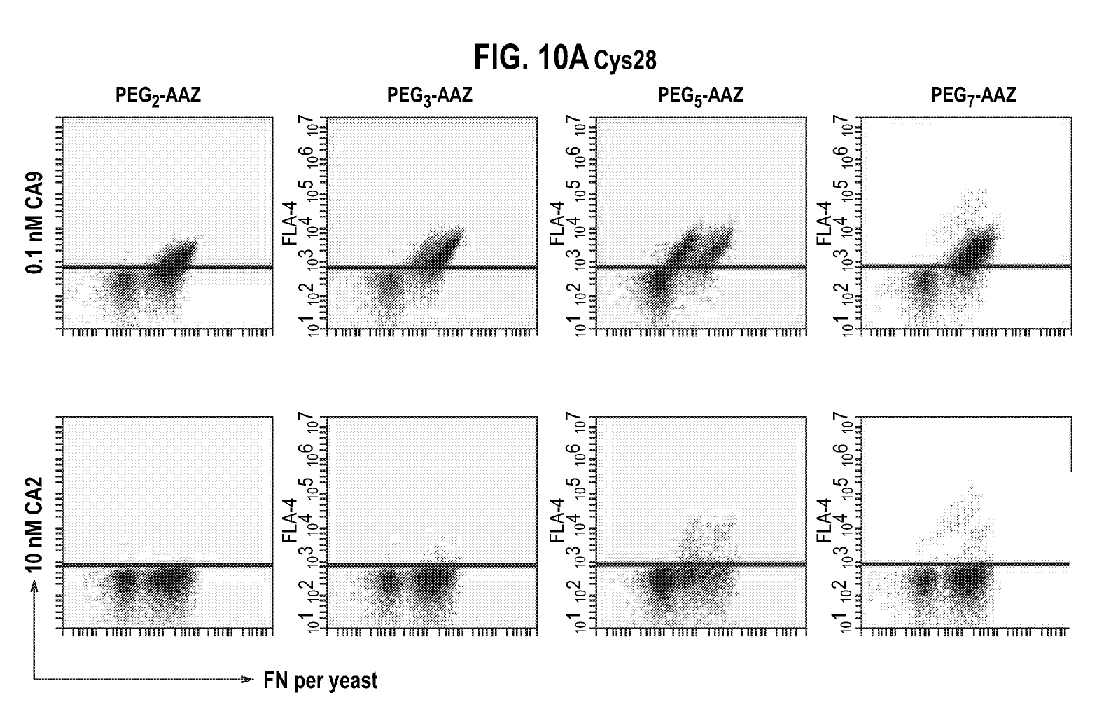

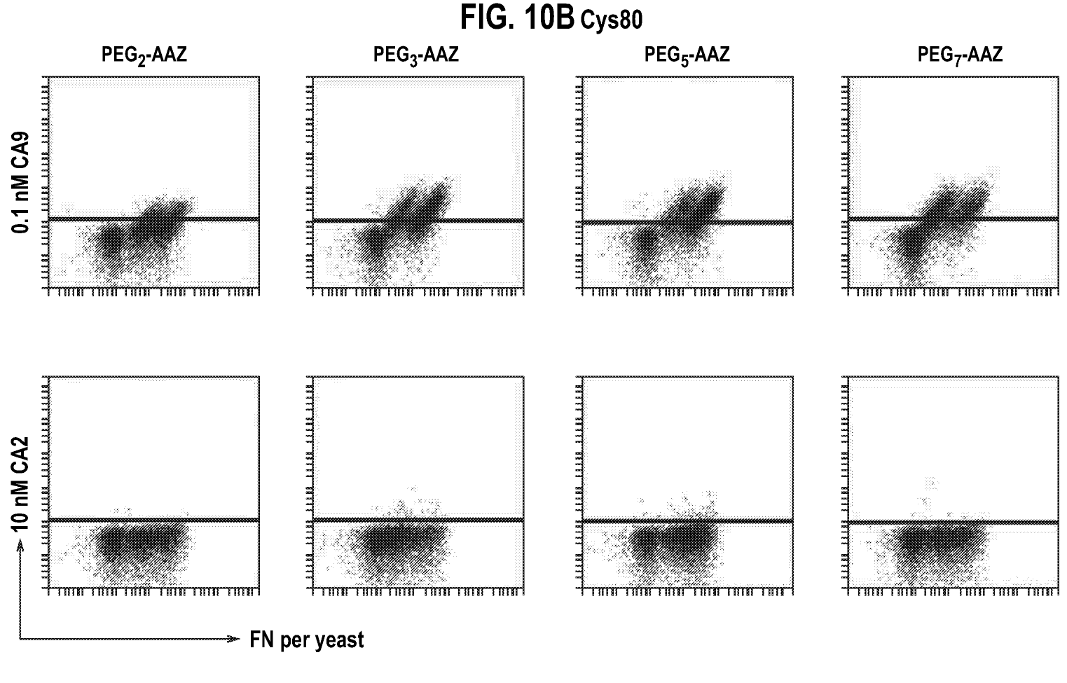

As shown in FIG. 10, Fn-AAZ clones selected for binding to carbonic anhydrase 9 were found to bind selectively to carbonic anhydrase 9 (CA9) over carbonic anhydrase 2 (CA2). Yeast-Fn-AAZ populations enriched for CA9 binding were induced to display Fn and conjugated with maleimide-PEG-AAZ and washed. Conjugated yeast were labeled with 0.1 nM biotinylated CA9 or 10 nM biotinylated CA2, washed, and labeled with AlexaFluor647-conjugated streptavidin. Yeast were also labeled with mouse anti-c-MYC antibody and AlexaFluor488-conjugated anti-mouse antibody to identify full-length Fn in the library populations. CA9 specific binding is observed in both the T28C libraries and the D80C libraries.

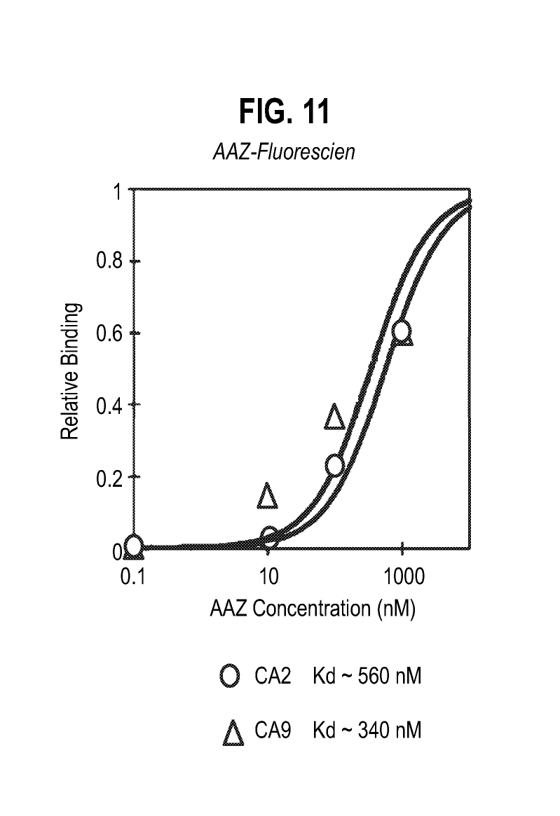

By contrast, FIG. 11, shows that, in the absence of conjugation to an Fn domain, AAZ-Fluorescein shows relatively little selectivity for CA9 (K_{d}=340 nM) over CA2 (K_{d}=560 nM).

Fn-AAZ can be Selected for Binding to Carbonic Anhydrase 2

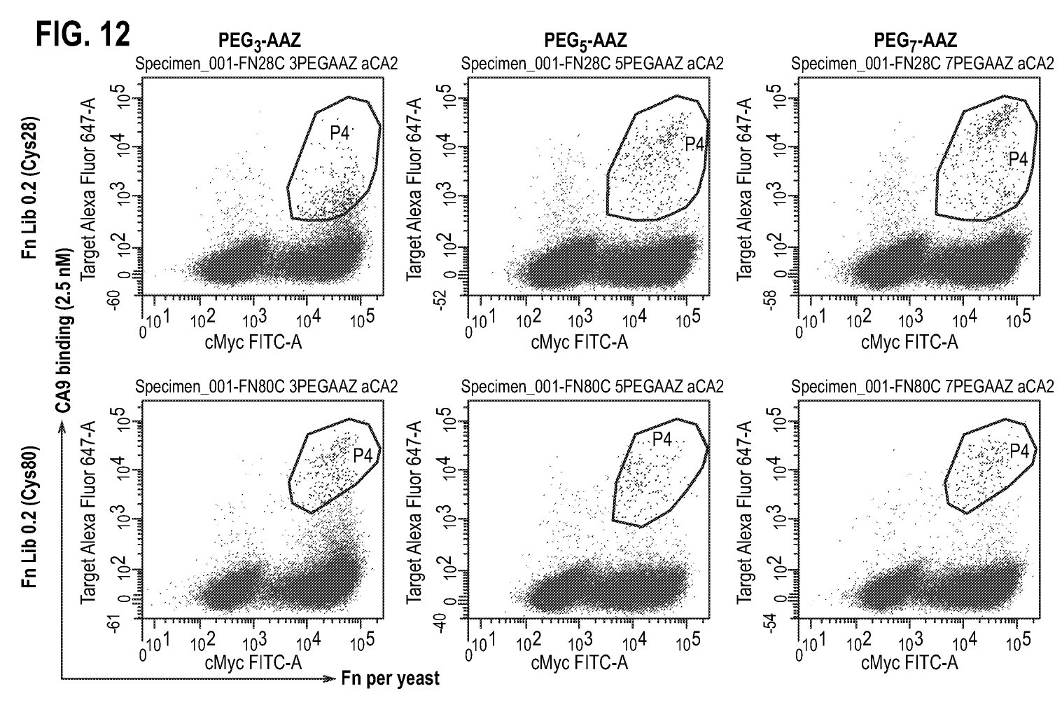

As shown in FIG. 12, libraries of yeast-displayed Fn-AAZ conjugates can be enriched to identify clones that bind with high affinity to carbonic anhydrase 2. Yeast-displayed Fn libraries were sorted twice for binding to carbonic anhydrase 2 (two bead sorts for PEG3 population; one bead sort and one flow cytometric sort for PEG5 and PEG7 populations). The resulting populations were conjugated with maleimide-PEG-AAZ. Yeast were labeled with 2.5 nM biotinylated carbonic anhydrase 2, washed, and labeled with AlexaFluor647-conjugated streptavidin. Yeast were also labeled with mouse anti-c-MYC antibody and AlexaFluor488-conjugated anti-mouse antibody to identify full-length Fn in the library populations.

Fn-AAZ Conjugate Clones Identified Having High Affinity for Carbonic Anhydrase 9

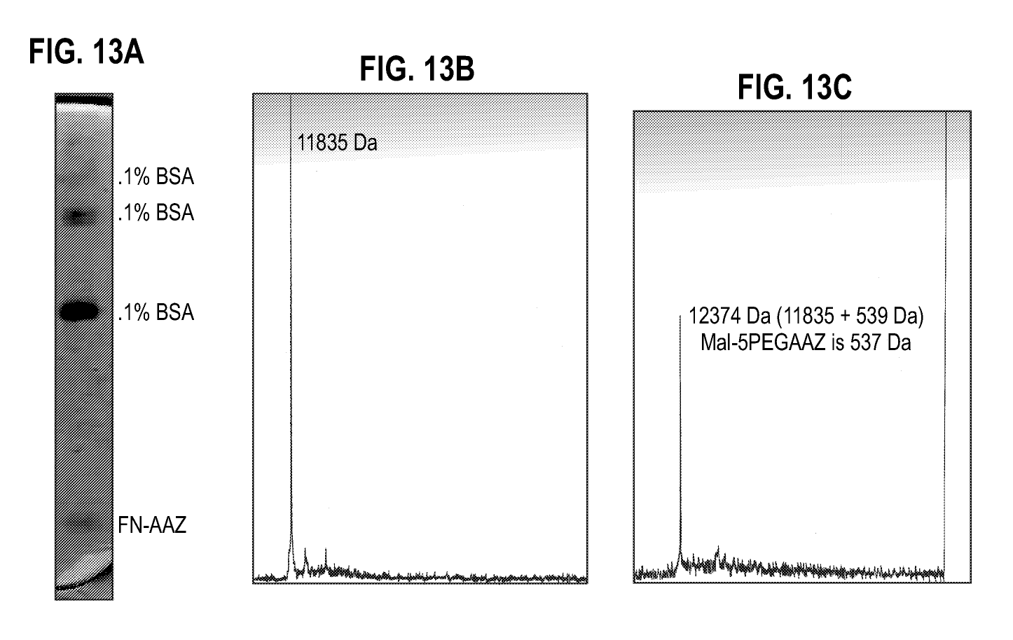

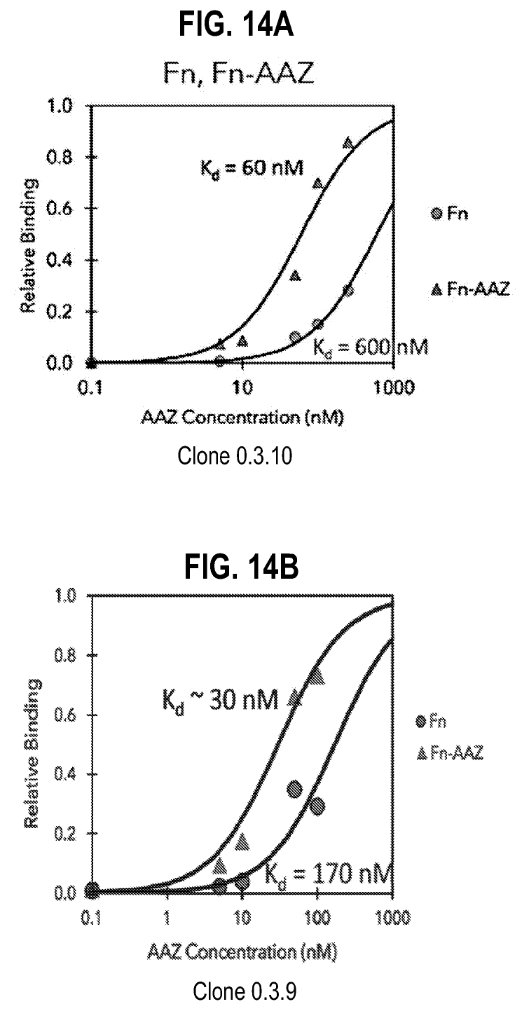

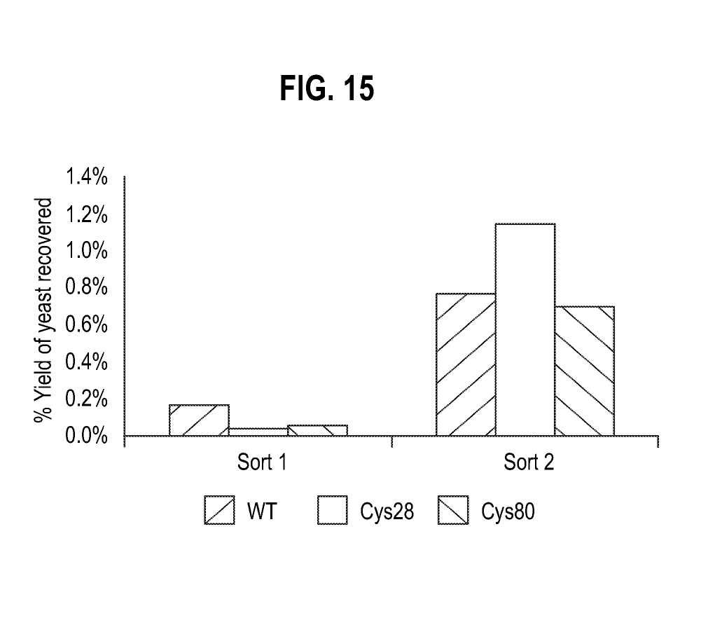

Fn-AAZ clones selected for binding to carbonic anhydrase 9 were expressed, purified, and sequenced according to methods well-known to one of skill in the art. FIGS. 13A-C show the results of an exemplary expression and purification of an Fn-AAZ clone. As shown in FIG. 14A-B, two Fn-AAZ conjugates were further characterized following expression and purification. A first Fn-AAZ clone (Clone 0.3.10) was identified that has 10-fold higher affinity to carbonic anhydrase 9 when compared to Fn that has not been conjugated to AAZ (FIG. 14A). The sequence of the selected fibronectin type III domain of the first Fn-AAZ conjugate corresponds to SEQ ID NO:3. A second Fn-AAZ clone (Clone 0.3.9) was identified that has approximately 6-fold higher affinity to carbonic anhydrase 9 when compared to Fn that has not been conjugated to AAZ (FIG. 14B). The sequence of the selected fibronectin type III domain of the second Fn-AAZ conjugate corresponds to SEQ ID NO:4. The sequences of the selected fibronectin type III domains of several other Fn-AAZ conjugates having high affinity and selectivity for carbonic anhydrase 9 are provided in SEQ ID NOs: 5-8.

SEQ ID NO: 3
MASSSDSPRNLEVTNATPNSLTISWDSYLDCAYYYRITYGETGGNSPSQE
FTVPGYTNSVTISGLKPGQDYTITVYAVASSNDVSNPISINYRTEIDKPS
QGS
SEQ ID NO: 4
MASSSDSPRNLEVTNATPNSLTISWDDSYCVIYYRITYGETGGNSPSQEF
TVPGYTNSVTISGLKPGQDYTITVYAVTDYSKLDPSNPISINYRTEIDKP
SQGS
SEQ ID NO: 5
MASSSDSPRNLEVTNATPNSLTISWDYHQNGCAVSYRITYGETGGNSPSQ
EFTVPGYYDTYSATISGLKPGQDYTITVYAVTGYNDDSNPISINYRTEID
KPSQGS
SEQ ID NO: 6
MASSSDSPRNLEVTNATPNSLTISWDYSYCVLYYRITYGETGGNSPSQEF
TVPGYYYSATISGLKPGQDYTITVYAVTDTGDESNPISINYRTEIDKPSQ
GS
SEQ ID NO: 7
MASSSDSPRNLEVTNATPNSLTISWDYSYCVLSYRITYGETGGNSPSQEF
TVPGYYTSATISGLKPGQDYTITVYAVATIDYKDSNPISINYRTEIDKPS
QGS
SEQ ID NO: 8
MASSSDSPRNLEVTNATPNSLTISLDDPSFCVIYYRITYGETGGNSPSQE
FTVPGYTNTATISGLKPGQDYTITVYAVASYGYLTSNPISINYRTEIDKP
SQGS

### Example 5. Identification of Bifunctional Compounds which Bind CXCR4

Libraries of bifunctional compounds were constructed by site-specific conjugation of a fibronectin type III domain (Fn), at either Cys28 or Cys80, to a cyclic peptide CXCR4 antagonist (CP) via a linker. The yeast-displayed Fn-CP libraries were screened for their ability to bind polypeptide target, CXCR4. FIG. 15 shows the % yield of yeast recovered following two rounds of FACS sorting of the yeast-displayed libraries of Fn-CPs. The resulting Fn-CPs are further enriched by additional rounds of sorting, and the resulting enriched populations are characterized for their ability to bind CXCR4, as previously described and by other methods known to one or skill in art. Individual Fn-CP clones are selected from the enriched population, and are further expressed, purified, and characterized for their ability to bind CXCR4 and related target.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any polynucleotide or protein encoded thereby; any method of production; any method of use) can be excluded from any one or more claims, for any reason, whether or not related to the existence of prior art.

1. A synthetic bifunctional compound, or a pharmaceutically acceptable salt thereof, that modulates the activity of an extracellular target protein, the compound comprising a polypeptide targeting moiety that binds to the extracellular target protein covalently conjugated to a small molecule moiety that binds to the extracellular target protein,
wherein the bifunctional compound binds to the target protein with at least 5-fold greater affinity and/or 5-fold greater selectivity than the affinity of each of the polypeptide targeting moiety and the small molecule moiety alone.

2. The synthetic bifunctional compound of claim 1, wherein the polypeptide targeting moiety is an antibody or an antigen binding fragment thereof or a fibronectin type III domain.

3. The synthetic bifunctional compound of claim 2, wherein the antibody or an antigen binding fragment thereof is an antibody or a fibronectin type III domain.

4-19. (canceled)

20. The synthetic bifunctional compound of claim 1, wherein the extracellular target protein is a carbonic anhydrase.

21. (canceled)

22. The synthetic bifunctional compound of claim 20, wherein the small molecule moiety comprises the structure of Formula II: 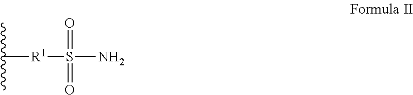
wherein R¹ is optionally substituted C₁-C₆ alkyl, optionally substituted C₁-C₆ heteroalkyl, optionally substituted C₆-C₁₀ aryl, or optionally substituted C₂-C₉ heteroaryl.

23. The synthetic bifunctional compound of claim 1, wherein the extracellular target protein is a metalloprotease.

24. (canceled)

25. The synthetic bifunctional compound of claim 23, wherein the small molecule moiety comprises the structure of Formula III: 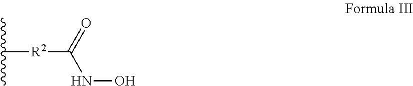
wherein R² is optionally substituted C₁-C₆ alkyl, optionally substituted C₁-C₆ heteroalkyl, optionally substituted C₆-C₁₀ aryl, or optionally substituted C₂-C₉ heteroaryl.

26. The synthetic bifunctional compound of claim 1, wherein the extracellular target protein is PSMA.

27. (canceled)

28. The bifunctional compound of claim 26, wherein the small molecule moiety comprises the structure of Formula IV or Formula V: 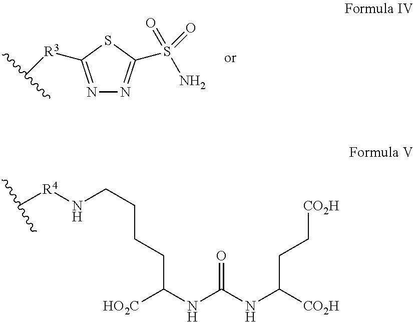
wherein R³ is optionally substituted C₁-C₆ alkyl or optionally substituted C₁-C₆ heteroalkyl (e.g., —NHC(O)—); and
R⁴ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

29. (canceled)

30. The bifunctional compound of claim 1, wherein the extracellular target protein is CXCR4.

31. (canceled)

32. The bifunctional compound of claim 30, wherein the small molecule moiety comprises the structure of Formula VI: 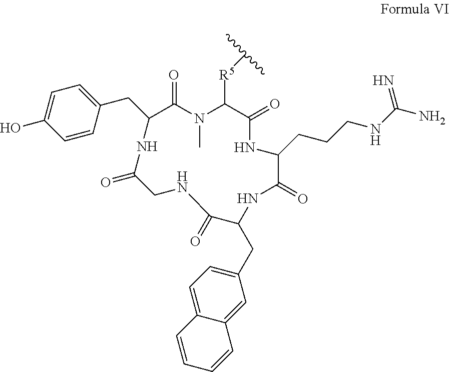
wherein R⁵ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

33. (canceled)

34. A method of identifying a compound that modulates the activity of a target protein, the method comprising:
(a) providing two or more synthetic bifunctional compounds comprising a polypeptide targeting moiety covalently conjugated to a small molecule moiety; and
(b) contacting a target protein with the two or more synthetic bifunctional compounds;
(c) determining the binding of the two or more synthetic bifunctional compounds to the target protein,
wherein a compound is identified as modulating the activity of the target protein if the synthetic bifunctional compound binds to the target protein with at least 5-fold greater affinity and/or 5-fold greater selectivity than the affinity of each of the polypeptide targeting moiety and the small molecule alone.

35-54. (canceled)

55. The method of claim 34, wherein the extracellular target protein is a carbonic anhydrase.

56. (canceled)

57. The method of claim 55, wherein the small molecule moiety comprises the structure of Formula II: 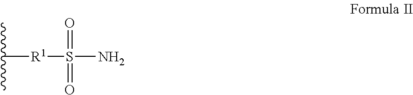
wherein R¹ is optionally substituted C₁-C₆ alkyl, optionally substituted C₁-C₆ heteroalkyl, optionally substituted C₆-C₁₀ aryl, or optionally substituted C₂-C₉ heteroaryl.

58. The method of claim 34, wherein the extracellular target protein is a metalloprotease.

59. (canceled)

60. The method of claim 58, wherein the small molecule moiety comprises the structure of Formula III: 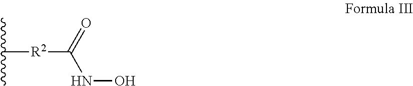
wherein R² is optionally substituted C₁-C₆ alkyl, optionally substituted C₁-C₆ heteroalkyl, optionally substituted C₆-C₁₀ aryl, or optionally substituted C₂-C₉ heteroaryl.

61. The method of claim 34, wherein the extracellular target protein is PSMA.

62. (canceled)

63. The method of claim 61, wherein the small molecule moiety comprises the structure of Formula IV or Formula V: 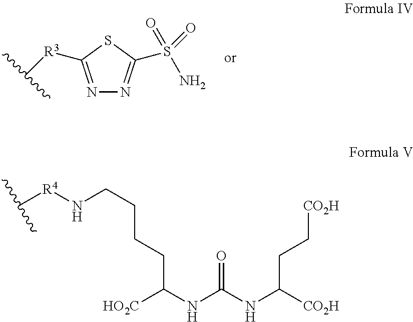
wherein R³ is optionally substituted C₁-C₆ alkyl or optionally substituted C₁-C₆ heteroalkyl (e.g., —NHC(O)—); and
R⁴ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

64. (canceled)

65. The method of claim 34, wherein the extracellular target protein is CXCR4.

66. (canceled)

67. The method of claim 65, wherein the small molecule moiety comprises the structure of Formula VI: 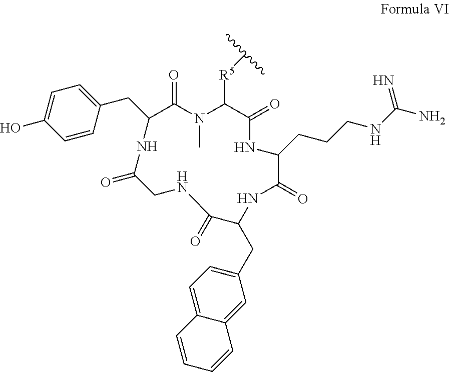
wherein R⁵ is optionally substituted C₁-C₆ alkyl, optionally substituted C₁-C₆ heteroalkyl, optionally substituted C₆-C₁₀ aryl, optionally substituted C₂-C₉ heteroaryl, or optionally substituted C₆-C₁₀ aryl C₁-C₆ alkyl, or optionally substituted C₂-C₉ heteroaryl C₁-C₆ alkyl.

68-73. (canceled)


SEQUENCE LISTING (8 sequences)

SEQ ID NO: 1 - protein, 94 aa, Artificial Sequence
  Type III domain of human fibronectin
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 81 aa, Artificial Sequence
  Type III domain of human fibronectin
  misc_feature 24: Xaa is any amino acid (2 or more of any amino      acid)
  misc_feature 48: Xaa is any amino acid (2 or more of any amino      acid)
  misc_feature 69: Xaa is any amino acid (2 or more of any amino      acid)
VSDVPRDLEVVAATPTSLLISWDXMYYRITYGETGGNSPVQEFTVPGXNATISGLKPGVDYTITVYAVXKSSKPISINYRT

SEQ ID NO: 3 - protein, 103 aa, Artificial Sequence
  Type III domain of human fibronectin
MASSSDSPRNLEVTNATPNSLTISWDSYLDCAYYYRITYGETGGNSPSQEFTVPGYTNSVTISGLKPGQDYTITVYAVASSNDVSNPISINYRTEIDKPSQGS

SEQ ID NO: 4 - protein, 104 aa, Artificial Sequence
  Type III domain of human fibronectin
MASSSDSPRNLEVTNATPNSLTISWDDSYCVIYYRITYGETGGNSPSQEFTVPGYTNSVTISGLKPGQDYTITVYAVTDYSKLDPSNPISINYRTEIDKPSQGS

SEQ ID NO: 5 - protein, 106 aa, Artificial Sequence
  Type III domain of human fibronectin
MASSSDSPRNLEVTNATPNSLTISWDYHQNGCAVSYRITYGETGGNSPSQEFTVPGYYDTYSATISGLKPGQDYTITVYAVTGYNDDSNPISINYRTEIDKPSQGS

SEQ ID NO: 6 - protein, 102 aa, Artificial Sequence
  Type III domain of human fibronectin
MASSSDSPRNLEVTNATPNSLTISWDYSYCVLYYRITYGETGGNSPSQEFTVPGYYYSATISGLKPGQDYTITVYAVTDTGDESNPISINYRTEIDKPSQGS

SEQ ID NO: 7 - protein, 103 aa, Artificial Sequence
  Type III domain of human fibronectin
MASSSDSPRNLEVTNATPNSLTISWDYSYCVLSYRITYGETGGNSPSQEFTVPGYYTSATISGLKPGQDYTITVYAVATIDYKDSNPISINYRTEIDKPSQGS

SEQ ID NO: 8 - protein, 104 aa, Artificial Sequence
  Type III domain of human fibronectin
MASSSDSPRNLEVTNATPNSLTISLDDPSFCVIYYRITYGETGGNSPSQEFTVPGYTNTATISGLKPGQDYTITVYAVASYGYLTSNPISINYRTEIDKPSQGS